(19) United States
(12) Patent Application Publication
(10) Pub. No.: US 2023/0393118 A1
(43) Pub. Date: Dec. 7, 2023

(54) DEVICES AND METHODS FOR MONITORING CELLS, TISSUES, OR ORGANS-ON-A-CHIP

(71) Applicant: University of Rochester, Rochester, NY (US)

(72) Inventors: John S. COGNETTI, Rochester, NY (US); Benjamin L. MILLER, Penfield, NY (US); Hani A. AWAD, Brighton, NY (US); Raquel E. AJALIK, Rochester, NY (US)

(73) Assignee: University of Rochester, Rochester, NY (US)

(21) Appl. No.: 18/034,693

(22) PCT Filed: Nov. 8, 2021
(86) PCT No.: PCT/US2021/058498

§ 371 Date: Apr. 29, 2023

Related U.S. Application Data
(60) Provisional application No. 63/110,971, filed on Nov. 6, 2020.
(60) Provisional application No. 63/111,054, filed on Nov. 8, 2020.

(51) Int. Cl. G01N 33/50; C12M 3/06; C12M 1/12; C12M 1/34; C12N 5/077

(52) U.S. Cl. CPC G01N 33/5044; C12M 23/16; C12M 25/04; C12M 41/38; C12N 5/066; G01N 33/5038; C12N 2533/54; C12N 2537/10; C12N 2513/00

(57) ABSTRACT

In some embodiments, the invention provides tissue-on-a-chip and organ-on-a-chip devices with integrated, on-board photonic integrated circuit optical sensors that allow real-time detection of analytes released from cells disposed on either side of a porous, ultrathin membrane within the device. The invention further provides modular devices for studying cells and interactions between and among cell types. The devices and methods using them are useful for, among other thing, modeling the biological and physiological interactions of cells of different tissue types, allowing high-throughput screening of drug candidates, and informing safety and efficacy in a virtual clinical trial.

### CROSS-REFERENCE TO RELATED APPLICATIONS

This application claims the benefit of U.S. Provisional Patent Application Nos. 63/111,054, filed Nov. 8, 2020, and 63/110,971, filed Nov. 6, 2020, the contents of which are incorporated herein by reference for all purposes.

### STATEMENT OF FEDERAL FUNDING

This invention was made with government support under 1UG3TR003281-01, awarded by the National Center for Advancing Translational Sciences of the National Institutes of Health. The government has certain rights in the invention.

### BACKGROUND OF THE INVENTION

For decades, scientists have studied biological functions by use of cell cultures in in vitro systems, followed by more expensive, but usually more informative, studies in animal models. Unfortunately, both cell culture systems and animal models have significant drawbacks. Cell culture systems typically use primary patient samples or cell lines of a single cell type, and by their nature are incapable of recapitulating the interactions between cell types in an organ. Animal models are not only costly, making it hard to scale them for screening, but can give information irrelevant for humans due, for example, to differences in enzymes or pathways in the animal used for the model and those present in humans. The problems presented by such systems contribute to, among other things, the loss of hundreds of millions of dollars in preclinical development and testing of potential therapeutics that then fail in clinical trials.

A decade ago, Huh et al. reported the development of a hybrid approach that could provide information at the level of a tissue or organ, as opposed to the cell level. See, Huh et al., Science, 2010, 328:1662-1668. Such “tissue chips” and “organs-on-a-chip” are microfabricated devices that support multicellular cultures of human cells interacting in microenvironments that more realistically resemble tissue. These tissue chip and “organ-on-a-chip” technologies (also referred to as “microphysiological systems,” or “3D cell culture”), are intended to fill some of the gaps between simple cell cultures and in vivo animal studies, addressing some of the deficiencies in both. For example, in 2015, Huh reported on developing a “lung-on-a-chip” model using “soft lithography-based microfabrication techniques to construct a compartmentalized three-dimensional microchannel system consisting of upper and lower cell culture chambers separated by a 10-μm-thick microporous elastomeric membrane made of poly-(dimethylsiloxane).” (Huh, Ann Am Thorac Soc. 2015; 12(Suppl 1): S42-S44.doi: 10.1513/AnnalsATS.201410-442MG). Human alveolar epithelial cells were seeded into the upper chamber and pulmonary microvascular endothelial cells were seeded onto the lower chamber and both types of cells were allowed to adhere to their respective side of the membrane. Huh reported that the system allowed investigation of the interplay between the different types of cells on the two sides of the membrane when one side was exposed to a stimulus, such as the introduction of proinflammatory cytokines. Id.

During the past decade that tissue chips and organ-on-a-chip systems have been available, they have been explored as alternatives for simple cell culture systems. While they represent an advance over single-layer cell cultures, however, several significant deficiencies have become evident.

In particular, there exists a lack of effective methods for analyzing the response of tissue chips and organ-on-a-chip systems to changes in their environment. Analysis is currently limited largely to methods such as lysing the cells on the chip or subjecting them to immunofluorescence microscopy Immunofluorescence-based assays, such as ELISAs, however, are irreversible by nature, meaning that once the measurement is taken, the experiment is over. Thus, deciphering time courses of analyte secretion or passage through the barrier used to constrain the cells on the chip requires many resources to repeat the experiment at each time point, increasing the time, cost, and variability of such studies. Tracking changes to the tissues or organ disposed on a chip requires multiple chips run in parallel so that a chip is available to be subjected to an experiment-ending analysis at each time point for which information is desired. It would be desirable to be able to assess the behavior of the organ-on-a-chip in real time in a nondestructive manner. While some investigators have tried to address this problem by integrating sensors substantially downstream of the system under study, this creates its own problems by decoupling the sensor from the organ-on-a-chip both temporally and spatially. Additionally, studies of cells cultured in current tissue chips is hampered by the fact that it is difficult to access the cells themselves.

It would be desirable to have methods and devices that allow assessing the effects on cells on a tissue chip or organ-on-a-chip that are non-destructive and that do not decouple sensors in time and space from the tissue chip or organ-on-a-chip. It would further be useful to have chips that can better simulate the forces on particular kinds of tissue than currently available tissue chips and organ-on-a-chip systems. And, it would be useful to have devices and methods that afford access to cells cultured in such chips and systems. Surprisingly, the present invention fulfills these and other needs.

### BRIEF SUMMARY OF THE INVENTION

In a first group of embodiments, the invention provides microfluidic devices for providing real-time information on analytes. The inventive devices of these embodiments comprise (a) a first microchannel fluidly connected to a port on an exterior of said device, and having a length, a first end, and a second end, (b) an ultrathin membrane having nanopores, mesopores, micropores, or a combination of two or more of these, said ultrathin membrane having a first side and a second side, wherein said first side of said membrane is fluidly connected through said first microchannel to said port on said exterior of said device, (c) a second microfluidic channel, which second microfluidic channel faces said ultrathin membrane and is fluidly connected to receive any fluid coming through nanopores, mesopores, micropores, or combinations thereof of said ultrathin membrane, and, (d) a first photonic integrated circuit sensor (“PIC sensor”) disposed in said first microchannel or in said second microchannel, which first PIC sensor is functionalized to detect the presence of a first analyte of interest in fluid in said first microchannel or said second microchannel, respectively. In some embodiments, the ultrathin membrane is a nanoporous membrane. In some embodiments, the ultrathin membrane is a mesoporous membrane. In some embodiments, the ultrathin membrane is a microporous membrane. In some embodiments, the ultrathin membrane has (a) a combination of nanopores and mesopores, (b) a combination of nanopores and micropores, (c) a combination of mesopores and micropores, or (d) a combination of nanopores, mesopores, and micropores. In some embodiments, the ultrathin membrane is of silicon nitride. In some embodiments, the first PIC sensor is disposed in said first microchannel. In some embodiments, the first PIC sensor is disposed in said second microchannel In some embodiments, the device further comprises a second PIC sensor, which second PIC sensor is disposed in said first microchannel or in said second microchannel, and is functionalized to detect the presence of a second analyte of interest in fluid in said first microchannel or said second microchannel, respectively. In some embodiments, the analyte said second PIC sensor is functionalized to detect the presence of is a control. In some embodiments, the device further comprises an outlet in said second microchannel to allow fluids in said second microchannel to exit the device. In some embodiments, the first PIC sensor is a photonic ring resonator. In some embodiments, the first PIC sensor is a photonic crystal, a spiral wave guide, or a Mach-Zehnder interferometer. In some embodiments, the second PIC sensor is a photonic ring resonator. In some embodiments, the second PIC sensor is a photonic crystal, a spiral wave guide, or a Mach-Zehnder interferometer. In some embodiments, the functionalization of said first PIC sensor is by covalently attaching to said first PIC sensor an antibody that specifically binds said first analyte of interest. In some embodiments, the first analyte of interest specifically bound by said antibody covalently attached to said first PIC sensor is a cytokine. In some embodiments, the device is configured to allow said first PIC sensor be exchanged by sliding said first PIC sensor out and sliding a fresh PIC sensor in. In some embodiments, the device is configured to allow said first PIC sensor be exchanged by opening said device, removing said first PIC sensor, and replacing it with a fresh PIC sensor. In some embodiments, the cells of a first cell type are disposed on said first side of said ultrathin membrane. In some embodiments, the cells of a second cell type are disposed on said second side of said ultrathin membrane. In some embodiments, the cells of a first cell type disposed on said first side of said ultrathin membrane are brain endothelial cells. In some embodiments, the cells of a second cell type disposed on said second side of said ultrathin membrane are pericytes, astrocytes, neurons, or a combination of any of these cell types. In some embodiments, the cells of a first cell type disposed on said first side of said ultrathin membrane are tendon fibroblasts. In some embodiments, the tendon fibroblasts are embedded in a hydrogel. In some embodiments, the device is configured to provide uniaxial stress to said tendon fibroblasts. In some embodiments, the device is “configured to provide uniaxial stress” is by vacuum actuators fluidly connected to a deformable wall of a space containing said hydrogel.

In a second group of embodiments, the invention provides methods for detecting if a first analyte of interest has been released from cells of interest or through an interaction between two or more types of cells of interest. The methods comprise (a) obtaining a microfluidic device comprising
(i) a first microchannel fluidly connected to an exterior of said device, and having a length, a first end, and a second end, (ii) an ultrathin membrane having nanopores, mesopores, micropores, or a combination of two or more of these, said ultrathin membrane having a first side and a second side, wherein said first side of said ultrathin membrane is fluidly connected to said first microfluidic channel, (iii) a second microfluidic channel, which second microfluidic channel is fluidly connected to said second side of said ultrathin membrane, and, (iv) a first photonic integrated circuit sensor (“PIC sensor”) fluidly connected to fluid in said first microchannel or said second microchannel, wherein said first PIC sensor is functionalized to change a detectable property of said first PIC sensor if a selected first analyte is present in fluid with which said first PCT sensor is in contact, thereby signaling said first analyte is present in said fluid, (b) disposing cells of a first cell type of interest on said first side of said ultrathin membrane, and, (c) allowing fluid in contact with said cells of said first cell type of interest on said first side of said ultrathin membrane to contact said first PIC sensor, and (d) detecting any signal from said first PIC sensor indicating the presence of said first analyte of interest in said fluid, thereby detecting whether said first analyte of interest has been released from cells of interest or through an interaction between two or more types of cells of interest. In some embodiments, the ultrathin membrane is a nanoporous membrane. In some embodiments, the ultrathin membrane is a mesoporous membrane. In some embodiments, the ultrathin membrane is a microporous membrane. In some embodiments, the ultrathin membrane has (a) a combination of nanopores and mesopores, (b) a combination of nanopores and micropores, (c) a combination of mesopores and micropores, or (d) a combination of nanopores, mesopores, and micropores. In some embodiments, the ultrathin membrane is of silicon nitride. In some embodiments, the methods further comprise step (b′) between steps (b) and (c): (b′) disposing cells of a second cell type on said second side of said ultrathin membrane. In some embodiments, the first PIC sensor is disposed on a layer in said device holding said ultrathin membrane. In some embodiments, the first PIC sensor is disposed in said first microchannel. In some embodiments, the first PIC sensor is disposed in said second microchannel. In some embodiments, the device further comprises a second PIC sensor, which second PIC sensor is functionalized to change a detectable property of said first PIC sensor if a selected first analyte is present in fluid with which said second PIC sensor is in contact, thereby signaling said first analyte is present in said fluid, wherein a signal from said PIC sensor indicates the presence of said second analyte of interest in said fluid. In some embodiments, the analyte said second PIC sensor is functionalized to signal the presence of is a control. In some embodiments, the first PIC sensor is a photonic ring resonator. In some embodiments, the first PIC sensor is a photonic crystal, a spiral wave guide, or a Mach-Zehnder interferometer. In some embodiments, the second PIC sensor is a photonic ring resonator. In some embodiments, the second PIC sensor is a photonic crystal, a spiral wave guide, or a Mach-Zehnder interferometer. In some embodiments, the functionalization of said PIC sensor is by covalently attaching to said first PIC sensor an antibody that specifically binds said first analyte of interest. In some embodiments, the first analyte of interest specifically bound by said antibody covalently attached to said first PIC sensor is a cytokine. In some embodiments, the cells of a first cell type disposed on said first side of said ultrathin membrane are epithelial cells or brain endothelial cells. In some embodiments, the cells of a second cell type disposed on said second side of said ultrathin membrane are pericytes, astrocytes, neurons, or a combination of any of these cell types. In some embodiments, the cells of a first cell type disposed on said first side of said ultrathin membrane are brain endothelial cells. In some embodiments, the cells of a second cell type are disposed on said second side of said ultrathin membrane and are pericytes, astrocytes, neurons, or any combination of pericytes, astrocytes, and neurons. In some embodiments, the cells of a first cell type disposed on said first side of said ultrathin membrane are tenocytes. In some embodiments, the tenocytes are embedded in a collagen hydrogel. In some embodiments, the device is configured to provide uniaxial stress to said tendon fibroblasts. In some embodiments, the device is “configured to provide uniaxial stress” is by having vacuum actuators apply a vacuum to said ultrathin membrane to cause said ultrathin membrane to stretch.

In another group of embodiments, the invention provides modular microfluidic devices. The modular microfluidic devices comprise: (a) a first module having a length, a width, a top, and a bottom, said first module comprising (i) a well or a first microchannel, said well or first microchannel fluidly connected to an exterior of said device, and, (ii) a ultrathin membrane having nanopores, mesopores, micropores, or a combination of two or more of these, said ultrathin membrane having a first side and a second side, wherein said first side of said membrane is fluidly connected to said bottom of said well or of said first microchannel, (b) a second module, having a length, a width, a top, and a bottom, wherein said top of said second module has a length and a width configured to mate with said bottom of said first module, said second module comprising a second well or second microfluidic channel fluidly connected to said top of said second module, and positioned to fluidly connect to said ultrathin membrane of said first module when said first module is placed on top of said second module. In some embodiments, the bottom of said first module has an exterior surface and said top of said second module has an exterior surface, wherein said exterior surface of said bottom of said first module and said exterior surface of said top of said second module are configured to contact each other when said first module is placed on top of said second module. In some embodiments, the exterior surface of said top of said second module bears an adhesive. In some embodiments, the adhesive is covered by a removable element. In some embodiments, the removable element is a protective film. In some embodiments, the well or microchannel in said second module has at least one crossbar spanning a dimension of said well or said microchannel. In some embodiments, the bottom of said second module is covered with a transparent material allowing viewing into said well or said microchannel of said second module. In some embodiments, the transparent material is cyclic olefin copolymer. In some embodiments, the ultrathin membrane is a nanoporous membrane. In some embodiments, the ultrathin membrane is a mesoporous membrane. In some embodiments, the ultrathin membrane is a microporous membrane. In some embodiments, the ultrathin membrane has (a) a combination of nanopores and mesopores, (b) a combination of nanopores and micropores, (c) a combination of mesopores and micropores, or (d) a combination of nanopores, mesopores, and micropores. In some embodiments, the ultrathin membrane is of silicon nitride. In some embodiments, the device further comprises an outlet in said second module allowing fluids in said device to exit. In some embodiments, the device has a first photonic integrated circuit sensor (“PIC sensor”) functionalized to detect presence of a first analyte of interest, which first PIC sensor is fluidly connected to said well, to said microchannel of said first module or to said well or said microchannel of said second module, or to both said well or said microchannel of said first module and to said well or said microchannel of said second module. In some embodiments, the first PIC sensor is a photonic ring resonator. In some embodiments, the first PIC sensor is a photonic crystal, a spiral wave guide, or a Mach-Zehnder interferometer. In some embodiments, the functionalization of said first PIC sensor is by covalently attaching to said PIC sensor an antibody that specifically binds said first analyte of interest. In some embodiments, the first analyte of interest specifically bound by said antibody covalently attached to said first PIC sensor is a cytokine. In some embodiments, the device further comprises a second PIC sensor functionalized to detect a second analyte of interest, which second PIC sensor is fluidly connected to said well or said microchannel of said first module, to said well or said microchannel of said second module, or to both said well or said microchannel of said first module, and to said well or said microchannel of said second module. In some embodiments, the second analyte of interest said second PIC sensor is functionalized to detect is a control.

### BRIEF DESCRIPTION OF THE DRAWINGS

FIGS. 1A-1E. FIG. 1A. FIG. 1A is an exploded view of an exemplar embodiment of a two-channel layered device incorporating both a nanoporous membrane and a photonic integrated circuit sensor chip, with alternating silicone and adhesive layers. FIG. 1B. FIG. 1B shows a schematic of the assembled device. FIG. 1C. FIG. 1C is a photograph of an exemplar assembled device. FIG. 1D. FIG. 1D is a phase contrast image of a monolayer of human bronchial epithelial cells. FIG. 1E. FIG. 1E is a phase contrast image of a monolayer of human cerebral microvascular endothelial cells. FIG. 1F. FIG. 1F is a trace of raw peaks corresponding to test (a-IL-6) photonic ring sensors and control (BSA) photonic rings sensors in response to IL-6 secreted from HBE cells cultured in the device pictured in FIG. 1C after being treated with lipopolysaccharide (LPS). Left: nonspecific binding to both rings as HBE cells are being exposed to LPS. Right: test ring peak shifts as IL-6 is secreted from cells and bound to functionalized ring. FIG. 1G. FIG. 1G shows subtracted shifts for IL-6 (left) and IL-1B (right) for a pair of control-test rings over the course of ˜3 hours. The increase beginning at about 70 minutes is due to secreted analytes being detected.

FIGS. 2A and 2B. FIG. 2A. FIG. 2A shows a chip functionalization schematic with two ring banks, with each ring bank consisting of five waveguides with two ring resonators each, and one bank with a single ring resonator plus an oxide-covered ring for use as a temperature control. The waveguides then return through output waveguides to a fiber array and detector. The shading in the two rows of ring resonators in the bottom panel shows the pattern of how different ring resonators in a study reported in the Examples were derivatized with different antibodies to demonstrate using the ring resonators for multiplex detection of a cytokine and an inflammatory biomarker. The notations superimposed over the rings denote which were derivatized with antibodies to FITC (control rings), with antibodies to C-reactive protein (“CRP”), or with antibodies to IL-1β. In this example, the chip is 4.4 mm wide×4.0 mm high. FIG. 2B. FIG. 2B is a graph of the response curves for IL-1β and CRP for a single chip under flow. Circles represent the results for IL-1β, while triangles represent the results for CRP. The Y axis on the left side shows the shift, in picometers, of the peaks from the rings functionalized with α-IL-1β, while the Y axis on the right side shows the shift, in picometers, of the peaks from the rings functionalized with α-IL-1β.

FIG. 3. FIG. 3, left side, shows an external view of an exemplar “tissue chip” embodiment of the inventive devices, while the right side presents an exploded view of the device showing the different layers and components. The exemplar device is 18 mm long, 10 mm wide, and 3.5 mm thick and is a human tendon-on-a-chip embodiment, containing tendon fibroblasts and, optionally, resident macrophages in a collagen hydrogel. A central channel containing the tendon hydrogel is flanked above and below by fluidic channels containing media, and on a far end by a flexible wall that applies load to the hydrogel by expanding and contracting in response to negative pressure in an adjacent vacuum chamber. A top acrylic housing is used to provide fluidic access to the device. The bottom layer is a glass coverslip, and all other layers are patterned from bioinert pressure sensitive adhesive (PSA), with the exception of the membrane spacer layer, which in this example is cut from silicone gaskets.

FIGS. 4A-B. FIG. 4A. FIG. 4A shows a proposed pathobiologic model and druggable targets in chronic inflammation and tendon fibrosis following tendon injury. FIG. 4B. FIG. 4b shows a schematic representation of an exemplar human tendon-on-a-chip (“hToC”) experimental setup to investigate the role of tissue-resident and circulating macrophages in activating the differentiation of myofibroblasts and the SASP-induced senescence by mTORC1 signaling in a tendon cell-collagen hydrogel on a membrane. Cyclic stretching force being applied uniaxially from the side is indicated by the wavy line on the right. The experimental setup typically includes a photonic ring sensor positioned below the second layer of cells to detect changes in analytes released by the cells; the sensor has been omitted in this representation for clarity of presentation.

FIGS. 5A-B. FIG. 5A. FIG. 5A shows the layout of an exemplar Photonic Ring Resonator (“PhRR”) chip. Each circle represents a ring resonator (195 μm) inside an etched oxide trench (300 μm). Each bus waveguide (depicted by a line coming from and returning to the right side of the chip) addresses a pair of ring resonators and light is coupled in and out via edge couplers at the right edge of the chip. FIG. 5B. FIG. 5B shows the layout of a second exemplar PhRR chip, with a different configuration of ring resonators, allowing the overall dimensions of the chip to be changed to accommodate the practitioner's need. As with FIG. 5A, each bus waveguide (depicted by a line coming from and returning to the right side of the chip) addresses a pair of ring resonators and light is coupled in and out via edge couplers at the right edge of the chip.

FIGS. 6A and 6B. FIG. 6A. FIG. 6A shows a schematic of the top section of an exemplar human tendon-on-a-chip (“hToC”) device. FIG. 6 B. FIG. 6 B shows a modification of the multilayer assembly in the schematic to accept a photonic sensor chip at one end in the same layer of the support chip as the ultrathin membrane. In this embodiment, the placement of the photonic sensor chip at the edge of the device enables facile coupling to an optical fiber array

FIG. 7. FIG. 7 is a drawing showing an embodiment of the exemplar human tendon-on-a-chip linked to multiple organ-on-a-chip devices intended to recapitulate not only the reactions of cells of the respective organs, but how flow of blood (or media) carrying analytes through one or more of the chips simulating different organs, tissue types, or one or more chips of each, can be analyzed by the inventive devices for biomarkers either at the level of the individual chip or after passing though one or more chips in the system of linked chips. The letters in circles are from the acronym ADMET (for Absorption, Distribution, Metabolism, Excretion, and Toxicity), and indicate which aspect of drug pharmacodynamics is being evaluated at each step.

FIGS. 8A-D. FIGS. 8A-D are drawings showing an exemplar modular microfluidic device, in this example, one with a top module, or “component,” and a bottom module. FIG. 8A. FIG. 8A is a top-down view of the top module of the device. The top module has a central well, with a square space at the bottom to accommodate an ultrathin membrane. Cells of interest can be cultured on the ultrathin membrane, which, when the top module is joined to the bottom component serves as the top channel In the embodiment shown, the bottom of the well is square, but the walls have wider cuts leading to the square bottom. The holes on either side of the well are ports extending through the top module that allow fluids to be introduced into the bottom module after the top module has been mated to the bottom module. FIG. 8B. FIG. 8B is a top view of the bottom module. An acrylic piece (gray area) has been cut to provide a bottom microfluidic channel. This exemplar device has two cross pieces, which are an optional feature for embodiments in which the cells to be cultured (such as tenocytes) grow better in a collagen hydrogel that can provide some support for the cell's tendency to contract. The bottom of the bottom module is sealed by a transparent, thin sheet of cyclic olefin copolymer (“COP”). FIG. 8C. FIG. 8C is a “ghost” image of the assembled device, with the top module pressed onto the bottom module, showing the ports in the top module fluidly connected to the bottom channel. FIG. 8D. FIG. 8D is an exploded view of the exemplar two module device showing the different structural layers, including the COP layer. The adhesive joining the top module to the bottom module is not shown.

FIGS. 9A-D. FIGS. 9A-D show results from studies conducted in the exemplar modular microfluidic device depicted in FIGS. 8A-D. FIG. 9A. FIG. 9A is a photograph of a cross-sectional view of an endothelial cell monolayer cultured in the top module and a fibroblast-laden collagen hydrogel in the bottom channel. The brightly lit cells in the upper layer shown the presence of vascular endotheial (“VE”) cadherin, while darker cells in the layer below are labeled with the fluorescent stain known as “DAPI” (4′,6-diamidino-2-phenylindole) or show the presence of actin. FIG. 9B. FIG. 9B is a photograph taken of a top view of the endothelial cell monolayer cultured in the top module. FIG. 9C. FIG. 9C is a photograph taken through the COP layer of the fibroblast-laden collagen hydrogel in the bottom channel FIG. 9D. FIG. 9D presents graphs quantifying the secreted cytokine profile of tenocytes that have been grown as a monoculture in the presence of TGF-β1 (“TC+TGF-β1,” represented in the graphs by diamonds,) or its absence (“TC-TGF-β1,” represented by upside down triangles in the graphs), as a co-culture with monocytes for 24 hours (“M/TC D1,” represented in the graphs by squares), for four days (“M/TC D4,” represented in the graphs by up-facing triangles), or for seven days (“M/TC D7,” represented in the graphs by dark circles), or as a tri-culture of monocytes, tenocytes, and endothelial cells (“M/TC/EC,” represented in the graphs by open circles) cultured for 48 hours. All of the cells were cultured in X-VIVO 10 medium, except for the M/TC/EC cells, which in this study were cultured in X-VIVO 10 medium supplemented with 10% fetal bovine serum (“10% FBS”). The letters and numbers above each graph identify the cytokine whose levels are reported in that graph. Asterisks indicate significant differences in cytokine levels (p<0.05; n=4−9). P>0.05 is not significant. *0.01<P<0.05. **0.001<P<0.01. ***0.0001≤P≤0.001. ****P<0.0001.

FIG. 10. FIG. 10 is a depiction of an embodiment of the top module of an exemplar modular microfluidic device in which the well in the top module has a photonic ring resonator chip extending into the well holding the ultrathin membrane.

FIGS. 11A and B. FIG. 11A. FIG. 11A is an exploded view of an exemplar microfluidic device configured to allow cyclic uniaxial stretching force to be applied to cells in a tissue chamber allowing cyclical stretching and releasing of the cells (the word “actuated” is used here to indicate that cyclical stretching force can be applied to the cells in the chamber). Between the actuated tissue chamber and the bottom microfluidic channel is a layer bearing both an integrated photonic sensor and a microporous ultrathin membrane, which fluidly connects the top microfluidic channel and the bottom microfluidic channel The end of the integrated photonic sensor chip bearing the bus waveguides extends beyond the edge of the device, allowing them to communicate with external instrumentation. “μSiM” indicates that the element of the figure depicted is a silicon-based microporous ultrathin membrane.

### DETAILED DESCRIPTION

As noted in the Background, “tissue chips” and “organ-on-a-chip” are hybrid systems that attempt to improve upon simple cull culture systems by modeling some of the biology of interactions between different cell types present in an organ of interest. These tissue chips and “organ-on-a-chip” (sometimes abbreviated as “OOCs”) systems also have the advantage of placing cells in a three-dimensional configuration that can better model the spatial relationship among the cells in the organ in which the cells naturally exist. Unfortunately, immunofluorescence-based assays such as ELISAs and other current techniques for analyzing the responses of cells present on current tissue chips and OOCs, typically require killing the cells, meaning that once the measurement is taken, the experiment is over. Thus, deciphering time courses of analyte secretion or passage through the model epithelial barrier keeping the cells on the chip requires many resources to repeat the experiment at each time point, increasing the time, cost, and variability of such studies.

Surprisingly, in some embodiments, the present disclosure provides devices and methods for not only non-destructively analyzing reactions of cells on a tissue chip or an OOC, but also doing so without separating the sensor analyzing the reaction of the cells from the cells temporally or spatially. Thus, in these embodiments, the inventive devices and methods allow analyzing the reaction of cells on a tissue chip or OOC over time and in response to one or more reagents without having to kill the cells and with unprecedented flexibility and ability to track changes in the cells or their activity in real time. The inventive devices with an integrated sensor are able to elucidate biological processes that were previously untestable, and to provide more timely and more sensitive elucidation of biological processes that were previously testable. The devices of this set of embodiments have a photonic integrated circuit, or “PIC.”

The present disclosure further surprisingly provides devices and methods for non-destructively analyzing reactions of cells of different types by providing modular systems in which cells can be incubated in separate containers (“modules”) that have been configured so that they can be joined together when desired by the practitioner to place the cells in the separate modules in fluid connection with each other across an ultrathin membrane, which has pores of a selected size range or ranges. The modular system embodiments make it easy for the practitioner to run parallel studies combining different cell types to elucidate the interactions among different cell types and to observe, for example, the effect of potential therapeutic agents on the different cells types combined in the modular device. In some embodiments, a PIC is disposed in the device, providing the benefits of real-time, sensitive detection of analytes as discussed regarding the devices described in the preceding paragraph.

The sections below explain different aspects of these different devices and methods. Then, the sections describe configuring several exemplar embodiments of the inventive devices and methods to demonstrate how the devices of the different types disclosed herein can be configured to provide different types of tissue chips and different types of organ-on-a-chip, or OOCs. In this regard, one section shows how to configure a device to provide an OOC with previously unobtainable capabilities. As the brain, with its blood-brain barrier, is hard to model with present techniques, it was chosen as the organ to use as an example to explain how to configure an OOC providing the benefits of some embodiments of the present invention. A second section describes how to configure a tissue chip using the information provided herein to provide previously unobtainable real-time information. Tendon was selected as the tissue to use as an example of how to configure a tissue chip using some of the teachings of the present disclosure. Other sections below describe modular embodiments, in which cells can be grown in separate modules and then joined together at a chosen time during a study, placing the cells in the previously separate modules in fluid communication with one another across a porous, ultrathin membrane.

### Integrated Devices, and Methods Using them, for Non-Destructive, Real-time Detection of Analytes

In some embodiments, the invention provides integrated devices and methods for the non-destructive, real-time detection of analytes released from cells. The analytes can be detected at a particular time or over time, as desired by the practitioner. The term “integrated” is sometimes used herein to describe the devices in this set of embodiments to mean they are provided as, and intended for use as, a single, integrated unit having a photonic integrated circuit (“PIC”), as opposed to the modular devices discussed elsewhere herein, which are designed so that cells can be first grown in physically separate containers, which are later combined to place them in fluid communication with one another.

As noted in the Background, tissue chips and OOCs typically comprise an in vitro microfluidic system with cells disposed on either side of a porous polymer membrane disposed across a chamber, or across a channel, thereby dividing the chamber into a first chamber and a second chamber, or dividing the channel into an upstream channel and a downstream channel. In contrast, the inventive devices use porous ultrathin membranes, as described further below. For convenience of reference, the discussion below will describe the porous ultrathin membrane as dividing a chamber into a first chamber and a second chamber, but it will be understood that the discussion also pertains to dividing a microfluidic channel into an upstream and a downstream channel. The discussion will also be couched in terms of cells being disposed above and below the porous ultrathin membrane in a vertical configuration. This configuration is convenient, because fluids typically pass from cells disposed above the porous ultrathin membrane, through the ultrathin membrane, to the cells adhering to the bottom of the membrane. It will be understood, however, that unless otherwise specified, the discussion also pertains to embodiments in which the ultrathin membrane is disposed vertically, with the cells disposed horizontally on either side of the vertical membrane. In some embodiments, the ultrathin membrane may be disposed at an orientation other than vertical or horizontal.

Unless otherwise specified, it is also understood that references to an ultrathin membrane refer to a membrane that is porous with respect to analytes of interest, but with pore sizes that are too small for the cells used in the tissue chip to pass through unless they actively migrate in response to chemotactic or other factors. As also discussed in the Background, up until now, there has been no way to monitor in real time changes to the cells on either side of the ultrathin membrane, as reflected for example, in the analytes they secrete, in response to environmental changes or challenges.

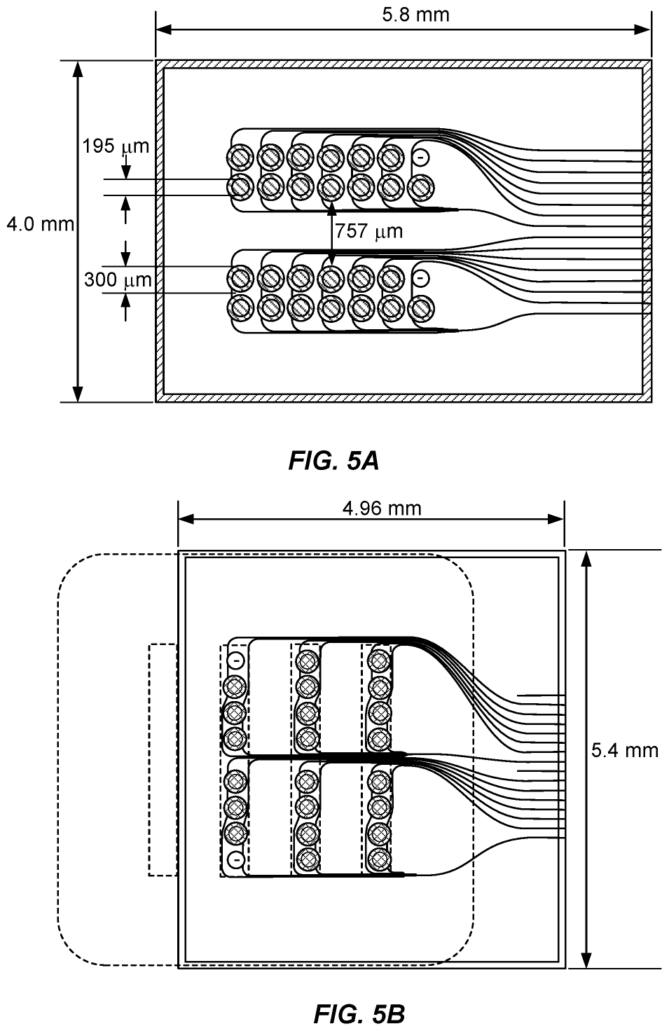

In one aspect, the integrated device embodiments of the invention solve this problem by incorporating into the devices PIC sensors that are sensitive, specific, label-free, and disposed in the fluid flow directly adjacent to cells of the simulated tissues or organs whose reactions are being monitored. For the first time, this enables the field to sense and quantify the passage of analytes through the barrier, or secretion of specific molecules by the cells in the device, in real time. Suitable PIC sensors and the practice of functionalizing them to detect different analytes will be discussed in a later section. For clarity, it is noted that the term “sensor” is used herein to denote the PIC sensors in configurations (including but not limited to trenches) directly exposing them to the fluidic milieu on a chip containing one or more such sensors. In a typical embodiment, the chip holding the sensors is 4.0 mm×4.4 mm (chips of other representative sizes are depicted in FIGS. 5A and B). As the chip holding the sensors in the inventive embodiments is typically present as one component of a multi-component “tissue-on-a-chip” or “organ-on-a-chip,” the chip holding the sensor or sensors will sometimes be referred to herein as a “sensor chip” to distinguish sensor-bearing chips from the overall tissue chip or OOC device containing the sensor chip as a component.

Additionally, the PIC sensors are capable of quantifying specific analytes in real time by monitoring the response of the sensor as a function of time. For a ring resonator, that means observing the spectral resonant peak in the spectrum, and how it changes over time. Typically, the sensors are “functionalized” by covalently attaching to the sensor an antibody or other molecule that specifically binds the analyte of interest to the investigator, or, for sensors intended for use as controls, specifically binds an analyte that is either not expected to be present or, in some cases, an analyte that is expected to be present in the experimental system, in which the failure to detect the analyte would indicate a problem in the experimental system. Any analyte whose presence the investigator wishes to be able to detect by use of a particular sensor is sometimes referred to herein as an “analyte of interest.” Binding of an analyte to the antibody or other specific-binding agent on the sensor causes a time- and concentration-dependent red-shift in the resonant peak, indicating the presence of the analyte in the fluid in which the sensor is in contact. Conversely, the absence of a resonant peak in the signal from the chip at any given point in time signals that the analyte of interest is not present in the fluid in which the sensor is in contact at that point in time.

The property of the sensors to provide a continuous read on the presence of one or more analytes of interest over a course of time allows using inventive devices incorporating this feature as a platform to elucidate biology that is difficult to obtain by current techniques. For example, cytokines are molecules secreted by cells that act as signals to other cells. There is considerable information available on the levels of various cytokines and other proteins in clinical serum and cerebrospinal fluid (“CSF”) samples, but very little is known about their concentrations in close proximity to the cells secreting them. Some studies have observed single-cell secretion of cytokines using label-free optical sensors. While data from clinical serum or CSF samples measures levels of cytokines on the order of 10-100 pg/mL using ELISAs, one study found the actual level in close proximity to the cellular source to be much higher, on the order of 1-3 ng/mL, decreasing to levels on the order of hundreds of pg/mL at relevant distances from the cell to the sensor units. Thus, this represents a more accurate sensing requirement for organ-on-a-chip models than clinical serum or CSF levels. As shown in the Examples, exemplar devices incorporating the PICs are able to capture information at the relevant levels.

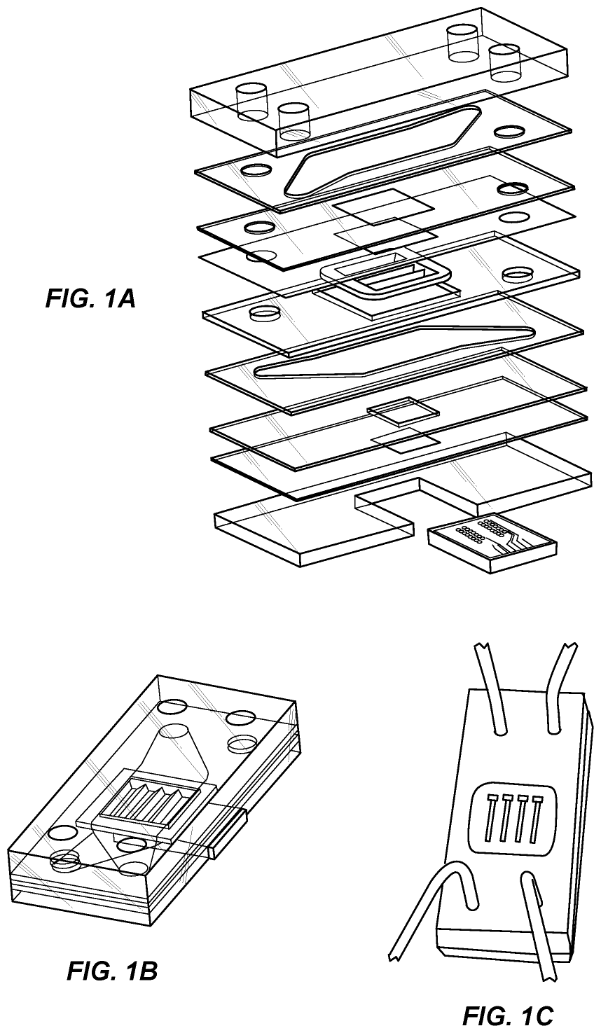

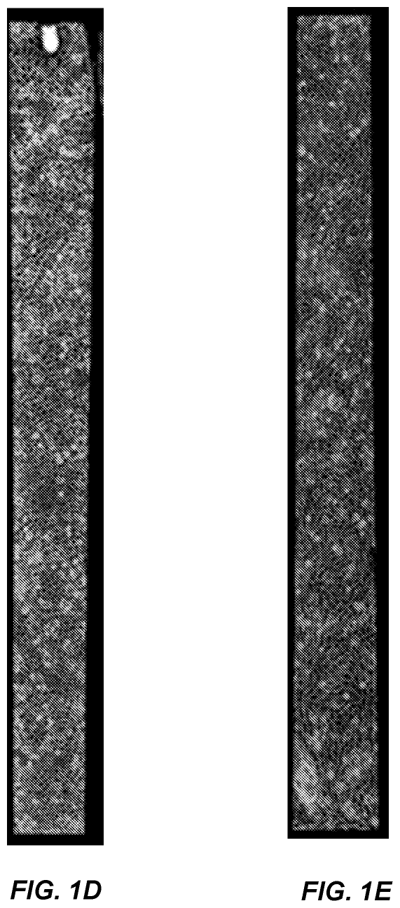

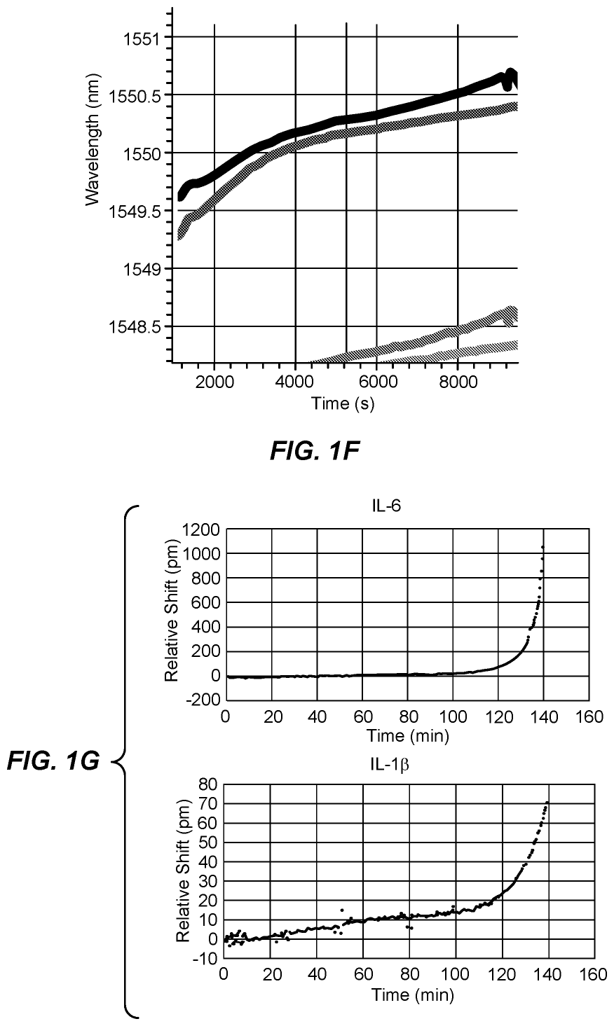

Some aspects of the inventive devices may be easier to understand in the context of a figure. FIG. 1A presents an exploded view of an exemplar embodiment of a two-channel layered device incorporating both a porous, ultrathin membrane and a PIC sensor chip. In a study reported in the Examples, the exemplar device was used to sense changes in refractive index due to diffusion of sucrose through the ultrathin membrane under flow conditions.

As persons of skill will appreciate, multi-layer microfluidic devices are built in layers “from the ground up.” Referring to FIG. 1A, and starting from the bottom, layer 1 is a silicone holder holding a photonic integrated chip sensor (the layer is 750 μm in depth); layers 2 and 3 are sealing layers (57 and 127 μm, adhesive and silicone, respectively); layer 4 is a “bottom” microfluidic channel for the organ system (127 μm in depth); layer 5 is a silicone holder holding an ultrathin membrane (300 μm); layers 6 and 7 are sealing layers (57 and 127 μm, adhesive and silicone, respectively); layer 8 is the top microfluidic channel, (127 μm); and layer 9 is a polydimethylsiloxane (“PDMS”) cap with inlet and outlet tubing (2 mm). FIG. 1B shows a schematic of the assembled device. FIG. 1C is a photograph of an exemplar assembled device. FIGS. 1D and E show human bronchial epithelial cells and human cerebral microcapillary endothelial cells, respectively, cultured on a nanomembrane with the device. FIG. 1F shows a representative raw peak trace from sensing of IL-6 secreted from cells cultured on an ultrathin membrane within a device of an embodiment of those described in this paragraph. Cells on the ultrathin membrane are stimulated with lipopolysaccharide (“LPS”). Approximately 70 minutes after LPS stimulation, the cells secrete IL-6 into the bottom channel, after which the IL-6 diffuses to the sensor. The sensor has “control” rings, which are not functionalized with an antibody that binds IL-6, and “experimental” or “test” rings which are functionalized with anti-IL-6 antibody. Initially, there is non-specific binding to both the control and the experimental rings, but as the secretion continues, the signal from the experimental rings shifts. FIG. 1G shows control-subtracted shifts for two sets of control and test ring pairs, one pair testing for IL-6 and another pair testing for IL-1β. Initially, differences in the nonspecific binding of the experimental and the control rings result in a negative relative shift, but as IL-6 and IL-1β secreted from cells on the membrane reaches the ring pairs of control rings and experimental rings functionalized with antibodies to IL-6 (FIG. 1G, top graph) or to IL-1β (FIG. 1G, bottom graph) respectively, the graphs show a relative shift.

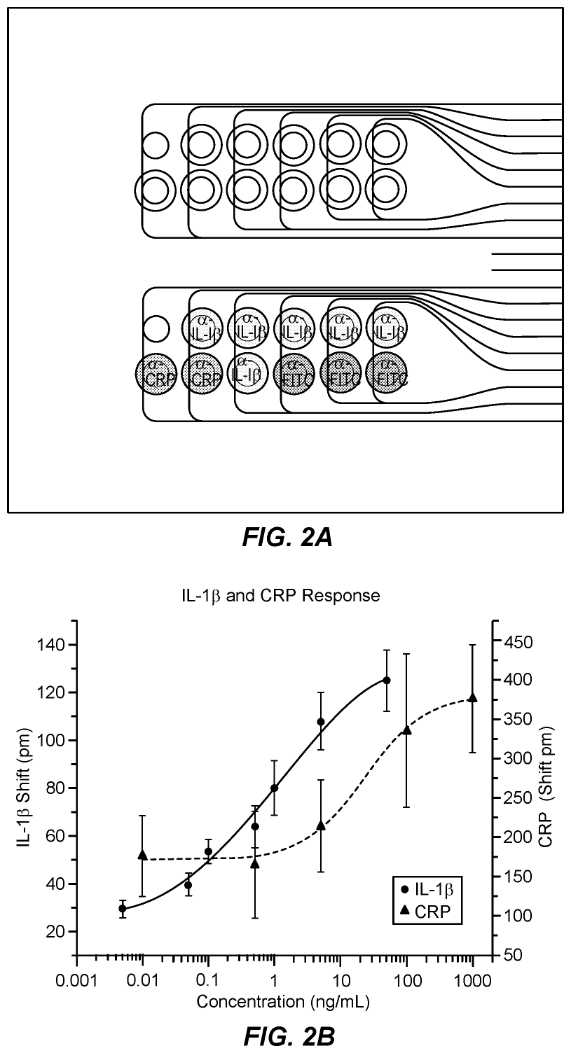

In some embodiments of the invention, the device can have a single PIC sensor. Preferably, however, the device has a plurality of sensors. In some embodiments, each of the PIC sensors is tuned to detect the presence of the same analyte. In preferred embodiments, however, at least some of the PIC sensors are tuned to detect a different analyte than that of some of the others to allow detecting multiple analytes at the same time, a capability known as “multiplexing” or “multiplex sensing.” An exemplar sensor chip is shown in FIG. 2A. FIG. 2A shows a chip functionalization schematic, with input waveguides split into six banks of 2 rings (sensors) each, which then return through output waveguides to a fiber array and detector. The bottom panel shows two rows of sensors used in a study reported in the Examples to demonstrate using the chips for multiplex detection of cytokines and inflammatory biomarkers. Referring to the bottom two rows of sensors in FIG. 2A, all of the sensors in the top row were functionalized to detect the cytokine IL-1β, while the left two sensors in the bottom row were functionalized to detect the biomarker C-reactive protein (“CRP”). The three sensors in the lower right of the bottom row were functionalized with α-FITC (fluorescein 5-isothiocyanate) as a control. In the embodiment depicted, the chip is 4.4 mm wide×4.0 mm tall×˜0.75 mm thick. Other embodiments can, of course, have chips of different sizes, and different configurations of sensors (for example, with more or fewer sensors per row or a different number of rows), depending on the size of the device on which it is to be integrated and the surface on which the sensors are disposed.

### Ultrathin Membranes and Pore Size Ranges

As noted above, in some embodiments, the inventive devices comprise a porous ultrathin membrane on which cells comprising the tissue chip or organ-on-a-chip can be disposed. Current membranes used in tissue chips and OOCs use thick, polymer-based membranes that limit barrier permeability. The porous ultrathin membranes used in preferred embodiments of the present invention provide significant advantages over the thick, polymer-based membranes in current tissue chips and OOCs.

Ultrathin (<400 nm thick) precision pore membranes have been made and their properties explored, as exemplified by Striemer, et al., Nature, 2007. 445(7129): p. 749-753; DesOrmeaux, et al., Nanoscale, 2014. 6(18): p. 10798-10805; and Winans, et al., J Memb Sci, 2016. 499: p. 282-289. Ultrathin membranes exhibit a unique combination of filtration properties. They are exceptionally permeable, enabling very low-pressure filtration in microfluidic devices

Ultrathin membranes have been made using pure silicon, silicon nitride, glass (SiO₂), MgF₂, gold, graphene, and various polymers. Because of their extreme thinness, ultrathin membranes are sometimes referred to as “2D membranes.” It is contemplated that ultrathin membranes made of any of the materials mentioned above can be used in the inventive devices and methods. In preferred embodiments, the ultrathin membranes are made of silicon, silicon nitride, silicon oxide, or silicon dioxide, as ultrathin membranes made of these materials are particularly robust. In some preferred embodiments, the ultrathin membrane is a silicon nitride ultrathin membrane.

Ultrathin membranes are so thin as to be transparent. They therefore better facilitate use of microscopy to monitor the device, for example to observe cells on the ultrathin membrane.

In some embodiments, the ultrathin membranes have nanopores (“nanoporous membranes”), which for purposes of this disclosure means it has pore sizes of ≤100 nm. In some embodiments, the ultrathin membranes have pores that are mesopores (“mesoporous membranes”), which for purposes of this disclosure means it has pores with sizes >100 nm but <1 μm. In some embodiments, the ultrathin membranes have micropores (“microporous membranes”), which for purposes of this disclosure means it has pore sizes ≥1 μm to 20 μm. In some embodiments, the ultrathin membrane has some pores that are nanopores and some that are mesopores or micropores, while in some embodiments, it may have nanopores, mesopores, and micropores. The pore sizes of ultrathin membranes for use in the inventive devices may be tuned by patterning them with pores of different sizes. For example, some of the pores on the ultrathin membrane may be nanopores and some may be mesopores or micropores. Alternatively, the ultrathin membrane may be provided with some pores that are mesopores and some that are micropores. Ultrathin membranes that are provided with pores of two or all three of the size ranges described above are sometimes referred to herein as “dual-scale membranes.” Selecting the size of the pores on the ultrathin membrane allows better control over the substances that can flow through the pores and, therefore, what reaches the sensor chips. For example, the pore size can be such as to allow analyte diffusion through the membrane, but not cells, or can be sized to allow cells such as monocytes to migrate from one side of the membrane to the other.

Cells of different types found in the tissue or organ of interest are placed on the chip on separate sides of the membrane to obtain information of interest to the practitioner about the interaction between the cell types in the tissue or organ. For example, in Huh et al. (Science, 2010, 328:1662-1668), lung epithelial cells were disposed on the upper side of a membrane, while endothelium cells were disposed on the underside (the membrane may be pretreated with factors from the extracellular matrix to encourage cells to adhere to the membrane despite being on the underside.)

### “On-Board” Photonic Integrated Circuit Sensor Chips

The inventive devices and methods utilize photonic integrated circuit sensors integrated into the devices themselves. By designing the devices with the sensors “on-board”, the sensors are much closer to the cells on the ultrathin membrane, allowing them to capture information about the presence or absence of analytes released or passing by the cells (i.e., not taken up by them) than that is both temporally and spatially closer to the cells than allowed by the use of prior chips, which have been positioned “downstream” of the tissue chips or OOCs, and thus further away from the cells of the tissue chip or OOC. Further, previous devices have used electronic sensors, but have not configured or adapted photonic sensors that can read the flow of analytes from tissue chips or OOC.

Photonic sensors have numerous advantages over electrical sensors as they do not require redox labels or other reagents to operate, can be fabricated inexpensively at scale using methods developed by the microelectronics and photonics industry, provide sensitive multiplex capability in a very small sensor footprint (down to a few square microns), and employ a measurement geometry in which the light source, sensor element, and detector are all nominally in the same plane, and therefore can be more easily integrated onto layered microfluidic devices than optical sensors incorporating free-space optics.

Ring resonators and photonic crystals use a combination of waveguide and configuration of the waveguide and associated structures that allows modulating the resonance of the sensor. Mach-Zehnder interferometers do not have a “resonance”, but report binding based on a change in phase. In embodiments of the inventive devices, the photonic sensor or sensors are derivatized with a molecule that binds (and preferably, specifically binds) an analyte of interest. For example, the molecule may be an antibody that specifically binds an antigen of interest, such as a particular cytokine or inflammatory biomarker. As another example, the molecule may be a receptor that specifically binds an enzyme that the practitioner wishes to monitor. When the cytokine or enzyme is present and binds to the antibody or the enzyme binds to the receptor, it changes the effective refractive index, signaling that the analyte has been detected. Methods of functionalizing photonic sensors to add antibodies, receptors, or other molecule that functions as an analyte-specific capture probe are known, as exemplified by Mudumba et al., J Immunol Methods, 2017, 448:34-43. An exemplar method is set forth in the Examples.

In a particularly preferred embodiment, the photonic sensors are photonic ring resonator sensors. As known in the art, ring resonator sensors comprise in relevant part a straight waveguide disposed immediately adjacent to a circular waveguide that serves as a ring resonator. Binding of the analyte to the antibody, receptor, or other molecule that functions as an analyte-specific capture probe on or in the immediate proximity of the waveguide changes the index of refraction and changes the resonant wavelengths in the ring resonator. Mudumba et al., supra, note that, as more material is deposited above the ring, the resonant wavelengths shift accordingly. As this shifts the color of the light resonant in the ring, it quantifies the amount of analyte of interest that has bound to the antibody, receptor or other capture molecule on the ring sensor. Changing the diameter of the ring allows the use of different resonance frequencies and makes multiplexing the sensors easy by sensing different analytes by rings of different resonant frequencies.

The small form factor (typically <200 μm diameter) of ring sensors allows for many sensor units to be incorporated on a single chip. Consequently, multiple analytes can be quantified simultaneously. In the sensors described by Mudumba et al., for example, 136 rings, each 30 μm in diameter, were etched on a 4×6 mm silicon chip, with 8 covered by a coating as controls and the other 128 rings organized in 32 clusters of 4 functionalized rings each exposed to the material flowing above them. The size of the particular sensors is selected to be suitable according to the desired resonant wavelength, taking into account the minimum bend radius of the particular material chosen for use as the substrate to hold the sensors. The minimum bend radius for materials, such as Si₃N₄ and Si, commonly used in the art to hold ring resonators, and sizing ring resonators according to the desired resonant wavelength and minimum bend radius, are well known in the art and it is assumed that practitioners can select ring sizes suitable for any particular material they choose to employ as a substrate.

High-Q micro-ring resonators are extremely sensitive to small changes in the refractive index environment above the chip. By modifying the top surface to selectively bind with particular biomolecules, they form the basis for sensing such changes. Working with AIM Photonics (Albany, NY), a national manufacturing institute, we have developed ring resonator designs demonstrating some of the highest sensitivities reported to date (Q=250,000; bulk refractive index sensitivity=253 nm/RIU) (“Q” is a “quality factor,” defined as the wavelength of the resonant signal divided by its linewidth at half-maximum). These devices are readily integrated with microfluidic channels, and, when derivatized with antibodies against proteins of interest, provide sensitive, quantitative detection in complex sample matrices such as serum or cell culture media.

In some embodiments, the devices use photonic sensors other than ring sensors. In some preferred embodiments, the photonic sensors other than ring sensors are photonic crystals. Two-dimensional photonic crystals (sometimes referred to herein as “2DPhCs”) are very small, and potentially allow the detection of single molecules of analytes. See, e.g., Joannopoulos, et al., PHOTONIC CRYSTALS: MOLDING THE FLOW OF LIGHT, 2^{nd} Ed. 2008 (Princeton Univ. Press, Princeton, NJ); Baker et al., Lab on a Chip, 2017, 17:1570-1577; Baker et al., Lab on a Chip, 2015, 15:971-990. In some embodiments, they are spiral wave guides. See, e.g., Densmore et al., Optics Letters, 2008, 33(6):596-598.doi.org/10.1364/OL. 33.000596. In some embodiments, they are Mach-Zehnder interferometers. See, e.g., Li, et al., Optics Express, 2012, 20(10):11109-11120. doi.org/10.1364/OE.20.011109. 2DPhCs consist of a periodic array of high refractive index/low refractive index materials. Easily fabricated in silicon-on-insulator (SOI) substrates, 2DPhCs confine light in a very small mode volume, yielding very high sensitivities (single particle) even for devices with a relatively low Q factor, where, as noted above, Q is defined as the linewidth at half height for the primary sensor resonance. Work in the labs of the present inventors has validated 2DPhC devices as highly sensitive sensors for proteins and virus particles in complex sample matrices such as serum. For example, a 2DPhC sensor was designed with several sensor cavities in series. In this device, introduction of a defect “hole” in the crystal produces a characteristic absorption in the transmission band. Since most of the light passes through the sensor, small variations in the defect size of sensors in series produce absorptions at different wavelengths. Tests done using a device functionalized with an anti-IgG antibody showed it could sense IgG, as a three-fold redundant detector for the protein. Similarly, we demonstrated that this sensor format could specifically detect virus-like particles (VLPs) from human papillomavirus (HPV) doped into fetal bovine serum (FBS). Sensor performance was similar in 10% FBS and buffer; this indicates that the device, coupled with appropriate surface chemistry and blocking methodology, is capable of rejecting nonspecific binding. Finally, a “large defect” structure (where the defect “hole” is matched to the size of a large particle) demonstrated recognition-mediated capture and detection of single particles under fluidic flow. This highlights the exceptional sensitivity of these structures. While photonic crystals are tiny (roughly 10×10 microns), and sensitive to the single particle or (potentially) to the single-molecule level, they are more subject to manufacturing variability than are ring resonators.

### Exemplar Blood-Brain Barrier OOC

The blood-brain barrier (“BBB”) plays an integral role in brain homeostasis. It protects the brain from toxins and pathogens, and its disruption in disease and injury leads to many problems, including immune dysfunction in the brain. In vivo BBB disease and injury models are expensive, labor intensive, incur ethical costs associated with the use of animals, and often do not translate well to human systems. Also, the use of animals or clinical subjects results in significant heterogeneity, lack of agreement between studies, and challenges in experimental throughput. This necessitates the development of in vitro models that replicate the complexities seen in humans in a way that is well controlled, as well as for testing neuropharmacological compounds in a high-throughput manner by reducing the use of animals and associated labor.

Alzheimer's Disease (“AD”) is one of the leading causes of death in America, and there are currently approximately 5 million people in the US living with AD. There is also a massive economic burden, which is expected to increase greatly in the coming years due to an aging population. AD consists of multiple pathologies, but the primary hallmarks of the disease are accumulations of both extracellular amyloid β plaques and intracellular neurofibrillary (tau) tangles in the brain. The buildup of these proteins also causes adverse immunological effects, including cytokine release and consequent chronic neuroinflammation, and blood-brain barrier dysfunction, ultimately resulting in the destruction of neurons and cognitive decline. Cytokines that are elevated in AD include IL-1β and IL-6, but the role of these and other cytokines in AD and the downstream effects they may have are unclear. Additionally, the many confounding variables in clinical samples have resulted in great variability in the quantity of these cytokines measured in cerebrospinal fluid (CSF) and blood in a number of clinical studies. These issues highlight the need for new approaches facilitating the study of cytokines in AD.

Current in vitro cell systems modeling the BBB utilize endothelial cells suspended on a permeable membrane, and incorporate sensing methods such as transendothelial electrical resistance (“TEER”) or fluorescence microscopy to determine barrier integrity. These methods allow for quantification of ionic flux or fluorescent markers on either side of the barrier, which gives a surrogate indication of the quality of the barrier. The best models also incorporate multiple cell types. While brain endothelial cells expressing tight junctions are a necessity, the inclusion of astrocytes and pericytes have also been shown to improve barrier integrity and match in vivo BBB characteristics more closely. Typically, microfluidics are used to provide the shear stress necessary to elicit barrier formation in dynamic models of the BBB.

Unfortunately, several technical constraints in these systems limit their utility. Most importantly, the lack of nondestructive specific analyte sensing represents a gap in the capabilities of current in vitro BBB models. In published models, any testing for specific analytes must be downstream of the device in the form of a labeled assay. As discussed above, immunofluorescence-based assays, such as ELISAs, are irreversible by nature, meaning that once the measurement is taken the experiment is over. Thus, deciphering time courses of analyte secretion or passage through the barrier requires many resources to repeat the experiment at each time point, increasing the time, cost, and variability of such studies.

Because of this, these time courses have yet to be elucidated. Also, the use of thick, polymer-based membranes limits barrier permeability.

To address these problems and to measure analytes in a label-free manner, antibody-functionalized photonic ring resonator chips are integrated into an OOC that simulates aspects of the blood-brain barrier (“BBB”). The BBB-OOC provides for the first time the ability to sense specific biomolecules in real time, in close proximity to the barrier model, yielding a platform useful for improving understanding of BBB dynamics in injury and disease.

Initial studies utilize the ability of nanoporous, ultrathin silicon nitride membranes to culture human bronchial epithelial (“HBE”) cells of the 16HBE cell line, as a barrier mimetic. After barrier properties are confirmed using immunohistochemistry and TEER, the barrier is chemically disturbed and the presence of analytes on either side of the barrier sensed using photonic ring sensor chips. Example 1, below, explains in more detail the construction and use of OOCs using on-board PIC sensor chips to provide real-time information on analytes in a model system using HBE cells.

Further, Example 2, below, explains the construction and use of OOCs using on-board PIC sensor chips to provide real-time information on analytes in a system for studying aspects of Alzheimer's Disease (AD), using human brain endothelial cells, on the “endothelial” side of the membrane, and human pericytes and, in some embodiments, astrocytes to the “brain” side of the nanoporous ultrathin membrane, with functionalized PIC sensor chips to monitor the effect of inflammatory proteins or potential therapeutic agents on the brain cells.

### Exemplar Tendon-on-Chip Tissue Chip Embodiments

In another aspect, the invention provides tendon-on-chip (“ToC”) systems for simulating clinical features of injuries to tendons, or of therapeutic agents on tendons. Use of human cell lines allows use of human tendon-on-chip (“hToC”) devices and systems.

The prevalence of musculoskeletal pathologies, including acute and chronic injuries, is higher than chronic circulatory and respiratory diseases, diabetes, and cancer. While not as fatal, musculoskeletal injuries have a substantial socioeconomic impact, with an annual medical care expenditure exceeding 160 billion, close to 1% of the national GDP. Second only to major surgery for joint replacement and spinal fusion, surgery to repair tendon and other soft connective tissues represent ˜20% (˜2.5 million) of all major musculoskeletal procedures. Minor procedures such as the injection of a therapeutic (e.g., steroids or platelet-rich plasma (“PRP”) into a joint or a tendon represent an additional ˜6 million procedures annually. Injuries to tendon/ligament can be acute, resulting from work-, sport-, or trauma-related full or partial tissue rupture, or can be chronic resulting from repetitive accumulation of microdamage due to overuse or aging, leading to a spectrum of painful, degenerative injuries collectively known as tendinopathy. The major injuries typically involve a variety of tissues including Achilles, Patellar, Quadriceps, Hamstring, Supraspinatus (rotator cuff), and hand and wrist flexor tendons.

Not surprisingly given the prevalence of tendon pathologies, there are over 250 currently active, interventional clinical trials of acute tendon injury and tendinopathy. The interventions include behavioral and physical therapy, surgical procedures, devices, drugs, and biologics (including cells and PRP). The primary outcomes in these studies include patient-reported outcomes, clinical scores, imaging of compositional and structural changes, and functional assessments (e.g. range of motion and strength).

Interestingly, none of these studies, including those evaluating potentially disease modifying drugs, use minimally invasive serum biomarkers as a primary outcome. We believe that the incomplete understanding of the pathobiology of tendon injury hinders the development of clinically validated, biologically relevant biomarkers, and that sophisticated human microphysiological systems (“hMPS”) can address this critical need.

Recent studies using next-generation sequencing and gene set enrichment analysis of injured flexor tendons of several mouse models correlated the improved tendon healing and reduced scarring with altered inflammatory, fibrotic, and cell cycle regulation, mediated by mTOR signaling, and an increase in serum levels of senescence-associated secreted proteins (“SASP”). These findings are consistent with published observations, which similarly associate peritendinous fibrosis in human patients with inflammation-induced fibroproliferative pathobiology. (Zheng, et al., J Adv Res. Jan 2019;15:49-58. Epub 2018/12/26).

A 3D multi-tissue human microphysiological system can validate these finding of pathobiology in tendon injury and the role of inflammatory mediators. Further, the induction of fibroproliferative and senescent phenotypes in our hMPS, can be ascertained using live microscopic imaging of α-SMA and γH2AX respectively, as well as any association with increased SASP. We also expect that real-time longitudinal measurements of SASP on the chip will identify select biomarkers that could be translated to clinical trials as primary biomarkers based on a blood test.

This disclosure provides a human Tendon-on-Chip (hToC) platform that simulates the clinical feature of fibrotic tendon scar, and in particular, the interactions between activated fibroblasts, inflammatory macrophages, endothelial vascular cells, and supporting extracellular matrix (ECM). The platform can use both human primary tendon fibroblasts and donor-matching human iPSC-derived macrophages and endothelial cells. Fluidic channels and ultrathin nano- and micro-porous membranes are used to make a multicompartmental device that enables paracrine signaling and cell migration through an endothelial cell barrier. The platform further enables confocal microscopy imaging and media sampling for quantitative assays. Mechanical actuation is integrated into the tendon side to cyclically load the tendon hydrogel.

Multiplex sensing of SASP associated with tendon injury and fibrosis is incorporated into the hToC and evaluate the limits of in situ detection. The sensing can be by antibody-functionalized Photonic Ring Resonator (“PhRR”) or 2D photonic crystals (“2DPhC”), both of which have been validated theoretically and experimentally, but have never previously been incorporated into a hMPS.

We hypothesize that mTORC1 signaling mediates fibrotic scar pathobiology, and as such, offers numerous druggable targets. The hToC serves as a virtual clinical trials platform by allowing dose escalation testing to be performed of existing FDA-approved mTOR inhibitors (Sirolimus and Everolimus) using donor tissues representing a spectrum of patients with fibrotic tendon pathology, as well as healthy controls. The primary outcome is SASP detection using the innovative, integrated sensor arrays. Secondary outcomes are based on microscopy evidence of myofibroblast activation and mTORC1 mediated senescence. The versatility of the hToC platform is demonstrated by performing a high throughput screen (HTS) of a 145 PI3K/AKT/mTOR Compound Library (APExBIO), including inhibitors and synolytics, to identify new anti-fibrotic drug candidates.

Integrating multiplex photonic sensing into the hToC system is a paradigm shift in the design of human microphysiological systems. Antibody-functionalized PhRR and 2DPhC are alternative systems for label-free sensing with high specificity and sensitivity. They enable on-chip multiplex sensing with the requisite sensitivities to measure biologically relevant secreted proteins over time at concentrations encompassing the range of values detected in blood serum, as well as damaged or diseased tissue. The hToC system models the complexity of the interactions between immune cells and tendon fibroblasts using a novel, 3D multi-compartmental design. The design employs ultrathin nano-, meso- or micro-porous membranes to enable the simulation of paracrine signaling and/or macrophage migration through an endothelial barrier to the tendon hydrogel, respectively. The membranes are highly permeable, optically transparent, and ultrathin (30 nm-300 nm), with precision pores sizes that can be tuned between ˜30 nm (to enable hormonal and paracrine signals) and 10 μm (to enable cell migration studies).

We expect our hToC system to meet a variety of criteria: first, to recapitulate the 3D physiological context (constrained aligned collagen ECM) of an injured tendon, with the collagen contracting only laterally. Second, to provide for the application of uniaxial cyclic stretch of a tendon hydrogel, with expectation that the tendon hydrogel will achieve a stretch of 1-5% without gel rupture or detachment. Third, to enable paracrine and cell-cell communication between macrophages and the tendon fibroblasts, with microscopic evidence of αSMA activation and senescence. Fourth, to incorporate fluidic channels to maintain the viability of the fibroblast-seeded collagen hydrogel, preferably with >90% viability. Fifth, it should incorporate vascular channels, where macrophages could be circulated, and endothelial barriers with nano- and micromembranes to simulate paracrine signaling and circulating macrophage infiltration of the hydrogel, respectively, with microscopic evidence of circulating macrophage infiltration of the hydrogel. Sixth, it should enable live and endpoint confocal microscopic imaging of the collagen hydrogel, with imaging of a 200 μm z-stack in the hydrogel. Seventh, and finally, it should enable fluidic sampling and ex situ analysis of senescence-associated secreted proteins (SASP), with ELISA-detectable levels of SASP (CXCL10, CCL2, CCL3, TNF-α, IL-1B, IL-6, IL-10, IL-17) in the range of 100 μg/ml to 100 ng/ml.

As noted in the second criterion in the preceding paragraph, in preferred embodiments, the hToC device is configured to provide uniaxial cyclic stretch. The ability to provide uniaxial cyclic stretch is sometimes referred to herein as “actuation.” In some exemplar embodiments, this is provided as follows. Cells of a type, like tenocytes, that have contractile properties are seeded in a hydrogel. The hydrogel containing the cells is placed on top of the ultrathin membrane of the device and is attached to a deformable “wall” on the long axis of the hydrogel (left and right, as the axis of the device is typically viewed along the long axis) through either surface modification or a horizontal anchor. The deformable wall is typically made of polydimethylsiloxane (“PDMS”) or another deformable polymer. The deformable wall is then deformed cyclically by placing it under a vacuum, which first pulls on the deformable wall and then turning off the vacuum, releasing the pull. Systems for placing cultures of cells under cyclic stretch, such as the Zoe® Culture Module (Emulate Bio, Boston, MA), are known, and it is assumed that practitioners can adapt such systems for use in embodiments of the inventive devices described herein.

Design and construction of an exemplar hToC device are set forth in the Examples, along with linkage of hToC devices with OOCs to provide pre-clinical information on likely absorption, distribution, and metabolism, and the use of hToC devices to screen compounds that are candidates for treating tendon fibrosis.

### Modular Devices

As noted above, in some embodiments, the invention provides modular microfluidic devices and methods allowing two or more sets of cells to be cultured in two separate containers, which containers (“modules”) are configured to be joined together when desired, with a fluid connection between the first container and the second container through an ultrathin, porous membrane, which may have pores that are nanoporous, mesoporous, microporous, or a combination of any two or of all three of these pore size ranges. The use of separate modules which can be joined together when desired allows the user to culture cells in a first container, optionally monitoring analytes of interest in the first container, and then place the second container on top (or vice versa, depending on which cells are in which containers and how the containers are configured) to monitor interactions between the cell types. For convenience of reference, the two containers will sometimes be referred to herein as the “top module” and the “bottom module,” as the top and bottom “components” or as the top and bottom “compartments”. The top module and the bottom module have internal surfaces which have been be etched or otherwise configured to define a microfluidic channel therein (a microfluidic channel in the top module is sometimes referred to herein as the “top microchannel” or the “first microchannel”, while one in the bottom module is sometimes referred to herein as the “bottom microchannel” or the “second microchannel.”

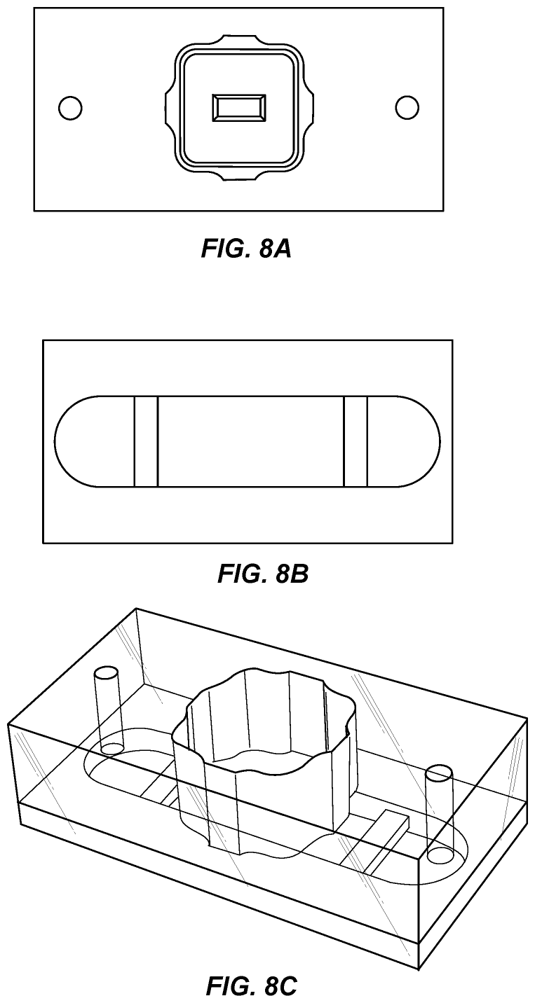

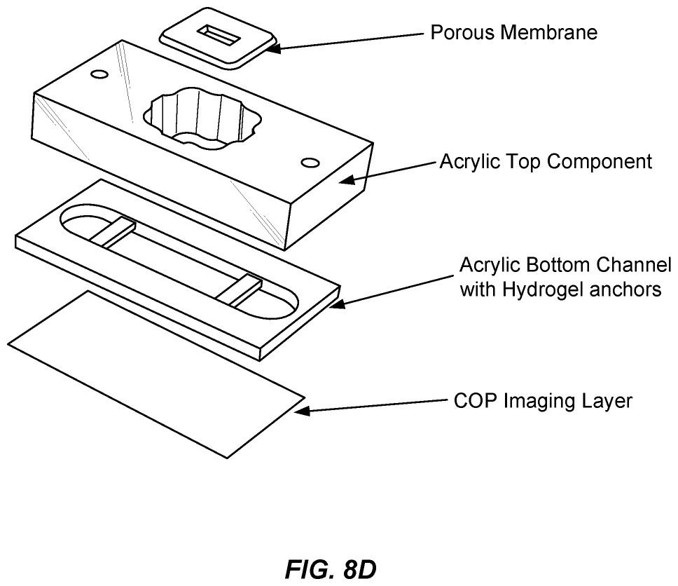

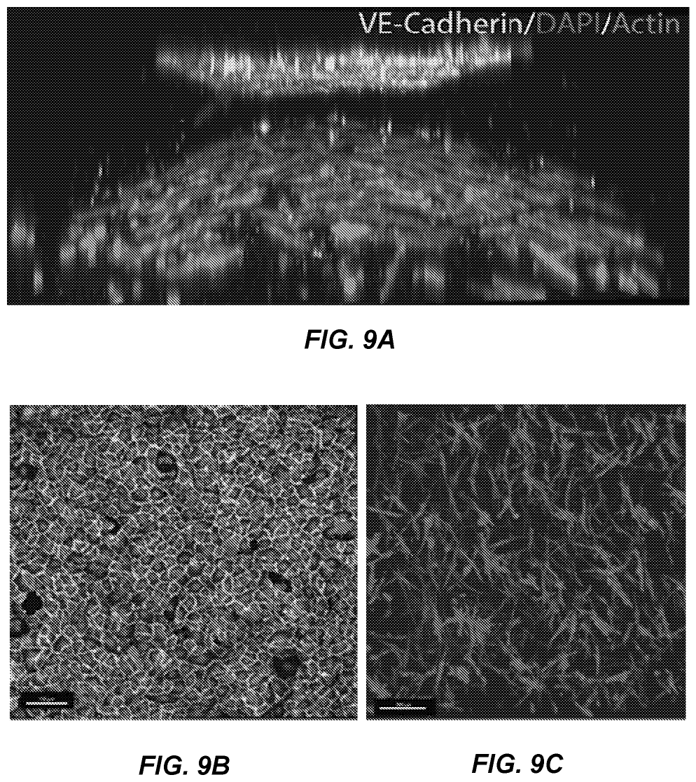

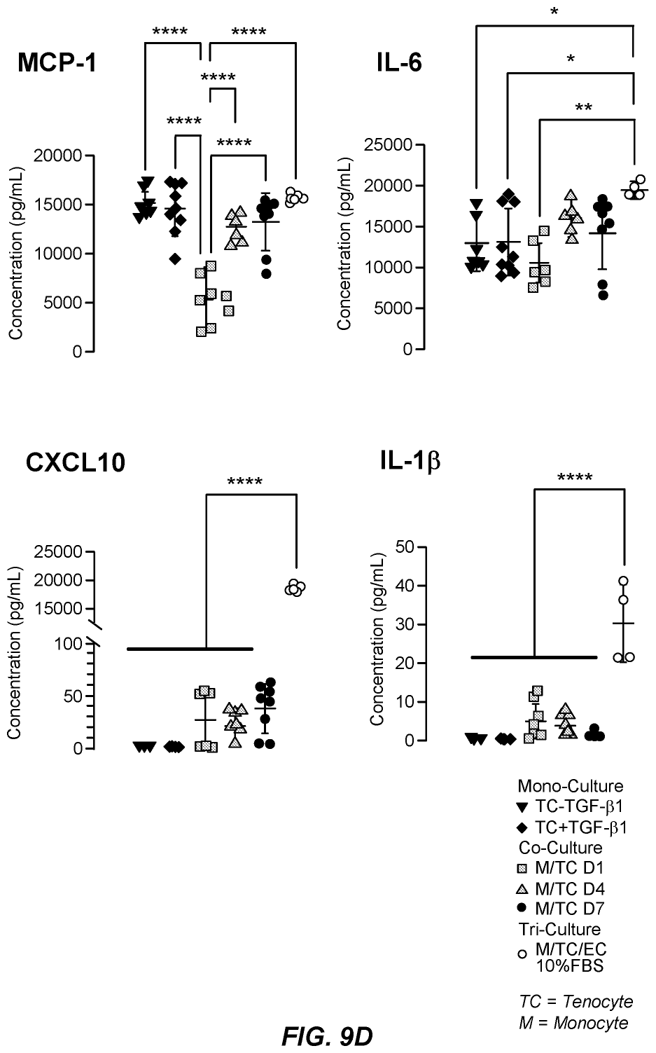

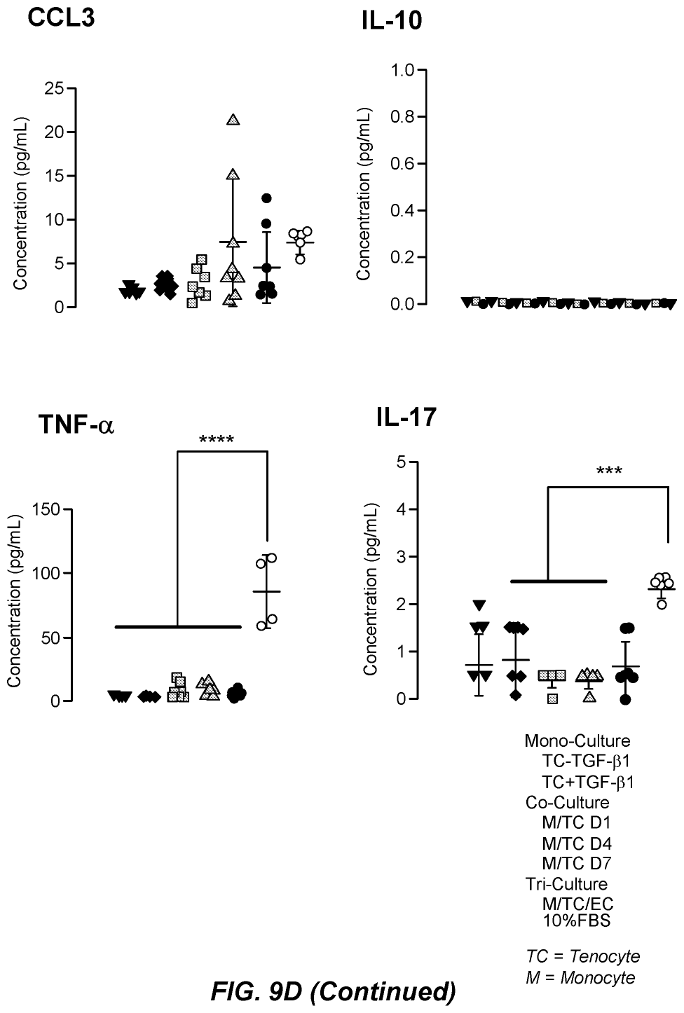

As noted, an ultrathin membrane with pores that are nanoporous, mesoporous, microporous, or a combination of any two or of all three of these pore size ranges is disposed between the cells in the top module and those in the bottom module. The choice of pore sizes allows the practitioner to control, for example, whether the membranes allow passage between the modules of only fluid containing soluble analytes, or of both fluid and cells, such as monocytes, that are capable of migration under appropriate stimulation. The ultrathin membrane can be in a space at the bottom of the top module configured to receive it and positioned so that media in the top module will pass through it before reaching the bottom module. FIG. 9A shows a top module with interior sides and a bottom defining a space to receive such an ultrathin membrane. Ultrathin membranes are typically shaped as squares. In the embodiment shown in FIG. 9A, the sides defining the space into which to insert the ultrathin membrane have cutouts forming spaces around the central square, allowing entry of tweezers or other fine tools used to introduce the ultrathin membrane into the module without breaking the membrane. In later studies, it was found to be more convenient to place the membrane on a “pedestal,” and to invert the top module and lower it gently over the membrane to position the membrane at the bottom of the top module. The bottom of the well in this embodiment has a hole in the middle sized to the length and width of the ultrathin membrane and has rim around the hole to retain the piece holding the ultrathin membrane within the well. Conveniently, the rim is provided with an adhesive facing up into the well before the ultrathin membrane is introduced into the well. When the ultrathin membrane comes into contact with the adhesive, the adhesive both prevents the ultrathin membrane from moving or falling out, and provides a seal around the ultrathin membrane so that the only fluid movement between the top compartment and the bottom compartment is through the pores of the ultrathin membrane or, in devices having them, inlet and outlet ports disposed to either side of the central well, as shown in FIG. 8C.

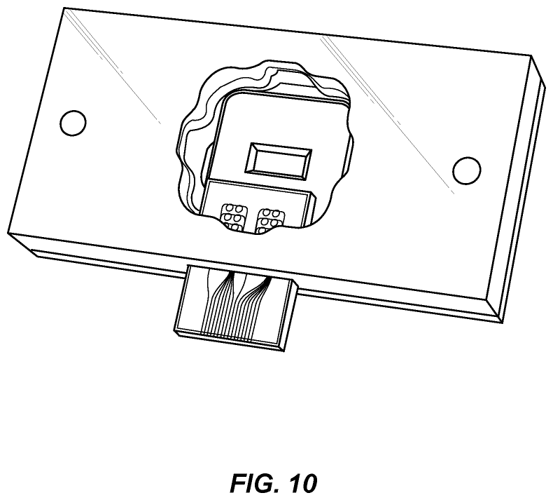

In some embodiments, a module of the modular device is further provided with an on-board photonic sensor, as shown in FIG. 10. In the exemplar embodiment shown, the photonic sensor is at the bottom of the top module next to the membrane. The chip bearing the sensors (in the embodiment shown, ring sensors) extends beyond the edge of the top module, allowing ready access to the connections coming from the ring sensors. The point at which the photonic sensor chip enters the top module may be provided with a deformable material, such as polydimethylsiloxane, with an opening, such as a slit, that is slightly smaller in dimension than the chip. When the chip is inserted through the deformable material into the module, it presses against the chip, providing a seal that prevents medium or other fluids in the top module from exiting around the chip.

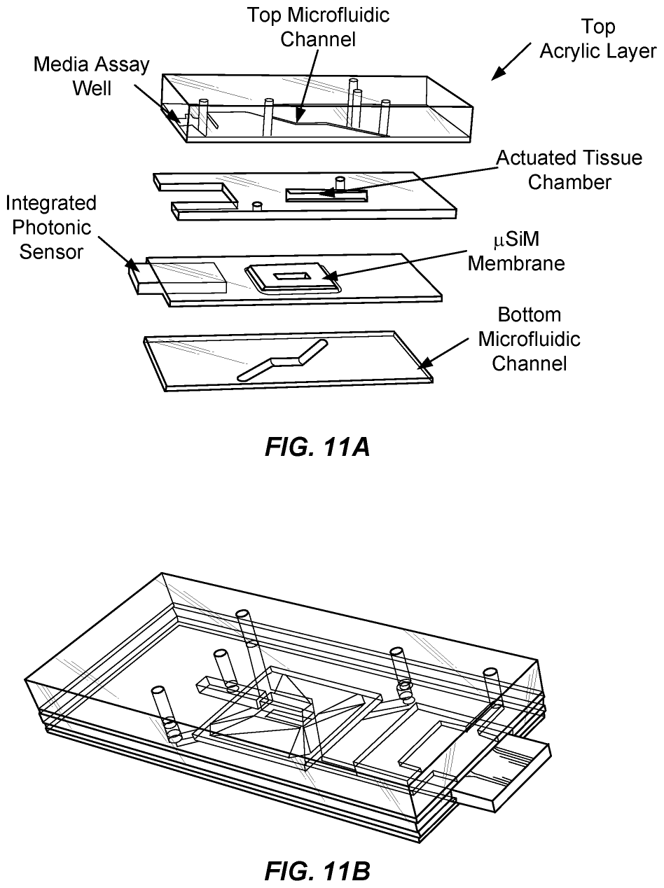

In another embodiment, depicted in FIG. 11A, the cells are disposed on a material deformable by uniaxial stretch, as described in the “Tendon-on-a-chip” section, and the membrane is disposed on a separate layer. The layer with the membrane may optionally also have a photonic sensor chip, as shown in FIG. 11A. The layers may be adhered by spot welds, an adhesive, or other methods known in the art. Preferably, the method used to adhere the layers together seal them together so that media placed in the microfluidic channels once the top module or bottom module is in the device does not seep out of the sides of the device when it is assembled.

### Embodiments Permitting Sensors to be Replaced

In some uses, as cells secrete an analyte to be detected by binding to antibodies functionalized on a sensor (e.g., a photonic chip), the sensor can become saturated by the analyte. Removing antigens bound to the sensor is typically performed by chemical regeneration, such as by applying HCl or a high salt concentration that allows the analyte to be eluted from the antibodies with which the sensor is functionalized. These traditional methods, however, can kill the cells disposed on the membrane above the sensor, terminating the experiment.

To avoid losing the ability to detect the presence of the analyte of interest, in some embodiments, the inventive devices are configured so that individual sensors, a selected group of sensors, or the entire array of sensors, can be removed and replaced, preferably without disturbing portions of the device holding other components. For example, the sensors can be disposed on moveable portion of the layer bearing the sensors, such as a tray or shelf that can be slid out of the layer in which it is disposed when the sensors need to be replaced. The sensors the practitioner wishes to replace can then be removed, fresh sensors inserted, and the tray or shelf, now bearing fresh sensors, slid back into the device. In another embodiment, the tray or shelf bearing the sensors can slide out of the device and be replaced by a fresh tray or shelf, bearing fresh sensors. The replacement tray or shelf is then slid into or snapped back into the device. In embodiments in which the tray or shelf is slid in and out a side of the device, the area on the side of the device through the tray or shelf slides can be covered with a deformable material, such as PDMS, that allows the tray or shelf to slide in and out of the device without allowing fluids in the device to seep from the device while the tray or shelf is in the device. It is contemplated that the “swap” or exchange of the sensors will take only seconds. If the exchange of an old tray or shelf for a new one is going to take any appreciable amount of time, a piece of tape or similar material can be placed over the area once the used tray or shelf has been removed to prevent loss of fluid from the device until the new tray or shelf is about to be put in position.

In an alternative embodiment, the device can be configured to open, for example, on a hinge along the long axis of the device, to expose the layer of the device bearing the sensors, making the sensors or the layer bearing them accessible for replacement. In these embodiments, a deformable material, such as PDMS, can be used on or around the hinge area to prevent fluid loss from that area. Devices designed to be opened have an upper portion and a lower portion, which, in an embodiment having a hinge along the long axis on one side of the device, are defined by the point at which the two hinged portions meet when the device is closed. The top and bottom portions preferably are configured to mate or to allow being sealed when the hinged device is in the closed position to avoid having fluid leak from the device. For example, the top portion or the bottom portion may have a lip that covers the other portion when the device is in closed position, which lip fits tightly enough to the other portion to block fluid from exiting around the lip. Alternatively, the top portion and the bottom portion may each be provided with a flat surface, which flat surfaces meet when the device is in closed position. The flat surfaces can be brushed with adhesive before the device is closed after the sensors have been exchanged to provide a seal keeping fluid from leaking around the area at which the two portions meet.

### EXAMPLES

### Example 1

This Example describes the construction and use of an exemplar device incorporating on-board photonic integrated circuit (“PIC”) sensors (“PIC sensors” or “sensors”) for an organ-on-a-chip modeling aspects of the blood-brain barrier (“BBB”), in particular, the tight junctions that limit movement of therapeutic molecules through the BBB into cells of the brain.

Silicon nitride nanomembranes are robust cell culture substrates for human brain-like endothelial cells. Mossu, J Cereb Blood Flow Metab. 2019; 39(3):395-410.doi: 10.1177/0271678X18820584. Epub 2018 Dec 19. Human bronchial epithelial (“HBE”) cells of the 16HBE cell line are a well-established human epithelial line, readily form tight junctions under culture, and have been studied extensively by our group for modeling cell barriers. These HBEs are cultured on a nanomembrane that has been integrated into the multichannel microfluidic device as described above. Barrier integrity is measured with TEER by incorporating semiconductor thin-film electrodes on either end of the microfluidic system (Masters et al., Nanomedicine, 2019, 2019;21:102039. Epub 2019/06/28), and the presence of tight junctions confirmed using immunofluorescent tagging of the tight junction proteins ZO-1, occludin, and claudin-1.

The device consists of two channels, separated by the epithelial barrier. The media of the top channel is doped with fluorescein isothiocyanate-dextran with a molecular weight of 40 kilodaltons (“FD40”), and a chemical agent to disrupt the barrier. FD40 is used because fluorescein isothiocyanate (“FITC”) does not interact with any human biological systems, and can be captured with anti-FITC antibodies, and visualized with fluorescence microscopy if desired. Synthetic tight-junction disrupting peptides (TJDPs) previously developed in our lab have been shown to reversibly disrupt tight junctions without toxicity in several cell lines (including the 16HBE cell line used in these studies), primary human cell cultures, and living mice. The FD40 that then passes through the disrupted barrier from the top channel is sensed and quantified by the photonic chip in the bottom channel. The photonic chips have 24 ring resonators. Two rings remain buried under oxide as a temperature control. Each chip has 4 rings functionalized with a negative control antibody (two per ring bank), such as anti-mouse IgG (“anti-mIgG”) or other commercially available antibody that does not react with human IgG) and up to 18 anti-FITC test rings. Resonance shifts are compared between the anti-FITC- and anti-mIgG-functionalized rings, and then averaged. This is repeated for at least three chip/barrier systems to achieve significance.

The depth of the channel between the membrane and the photonic chip affects the concentration of analyte at the sensors. The channel dimensions are designed to maximize the signal from proteins passing through the barrier, while allowing adequate flow through the channel and preventing interference from direct cellular contact with the PIC. This is a function of the diffusion rate of the analytes of interest under flow, as well as their anticipated concentration on each side of the barrier. These variables are modeled using the diffusion module in Comsol Multiphysics software. Comsol is also used to model the effects of varying flow rate on both sides of the barrier Channel dimensions and flow rates are balanced to accommodate in vivo capillary shear stress rates in the top channel as much as possible. Based on the modeling results, microfluidic dimensions and conditions are adjusted to optimize analyte concentration and capture.

For cell culture, cells must be kept at physiological temperature (37° C.) and receive a constant supply of nutrients and oxygen. To accommodate this, Flow EZ™ programmable microfluidic pumps (Fluigent Inc., North Chelmsford, MA) are used to constantly provide cells with media, while also controlling the flow of analyte-doped solutions through the device. Additionally, the Flow EZ™ unit pressurizes the media with gas containing 20% oxygen and 5% carbon dioxide, in accordance with standard cell culture protocols, and the media kept in a water bath at 37° C. Although the chip is closed to the environment and contamination or exposure is unlikely, all experiments are preferably done in a BSL-2-certified lab in the interest of safety. Cell health is monitored by viewing cell morphology upon each measurement under the microscope used aligning the device to an optical fiber array.

Experimental rigor and reproducibility: Sensing experiments are repeated on three devices to determine limits of detection for each analyte. Statistical significance is determined by performing one-way ANOVA for the shifts resulting from addition of each calibration concentration. Then an ANOVA is used to ensure there is not a significant difference between the concentration of a 50% maximum shift of each analyte between chips. Experiments are repeated until statistical significance is achieved.

Barrier integrity depends on the influence of many cell types and the factors they secrete. If barrier integrity is less than the current standards in the field, different culture media are tested to provide better stimulation of tight junction formation in the barrier. In particular, SF3 media has been shown to increase the expression of tight junction proteins, and will be used if necessary as a substitute media for coculturing additional cell types in this simplified system. Alternatively, or in combination with SF3 or other media, the flow rate over the cells may be increased, as that also increases barrier integrity.

Since this system relies on antibody-antigen binding to quantify the proteins of interest, some details about the degree to which antibodies on functionalized rings have become saturated must be understood. For increasing concentrations of secreted proteins, the signal is equilibrated to the new concentration of analyte. However, if the concentration of a given analyte decreases, the signal will not necessarily equilibrate immediately, according to the antibody's binding kinetics. To address this, the off-rate of the analytes is determined by introducing a known concentration of analyte, allowing the signal to equilibrate. The flow of analyte over the chip is then stopped the amount of time it takes for the signal to return to baseline is measured.

### Example 2

This Example describes the construction and use of an exemplar microfluidic device for modeling blood-brain barrier (BBB) pathophysiology in an in vitro model of Alzheimer's Disease.

Alzheimer's is a disease resulting in cognitive decline with age. The primary pathologies are the extracellular amyloid β plaques and intraneuronal neurofibrillary (tau) tangles in the brain. Additionally, the BBB is disrupted, resulting in chronic neuroinflammation. This includes the release of cytokines in the brain by brain endothelial cells and astrocytes, resulting in microglial activation, as well as their upregulation in peripheral circulation, and subsequent activation of peripheral immune cells. Currently, the exact cytokine profile and time course of cytokine secretion in the central nervous system in AD is unknown. Furthermore, it is unclear whether the role of each of these cytokines in BBB disruption is causative or secondary. With a multiplex BBB sensor chip, such as that provided by the present disclosure, it is possible to study the role of several inflammatory proteins in an AD model and their relation to BBB disruption for the first time.

It has been shown that the presence of Aβ, the primary extracellular toxin in AD, in the blood disrupts tight junctions in brain endothelial cells. In this model, the barrier must consist of brain endothelial cells rather than bronchial epithelial cells used in the model system discussed in Example 1. Cells of the hCMEC/D3 cell line will be used due to their established use in BBB models. Additionally, Aβ causes astrocytes and other cells to secrete cytokines, and so the barrier model also includes astrocytes and pericytes to observe this effect, as well as to improve initial barrier integrity. Finally, to study downstream neurodegeneration, neurons are incorporated in the “brain” channel of the device. Although no in vitro system can fully recapitulate the complex pathophysiology seen in AD in the human brain, this simplified model of AD allows addressing specific mechanisms underlying the complex clinical phenotypes seen in patients, while eliminating the heterogeneity that plagues clinical studies.

The in vitro BBB expands upon the initial model by sequentially adding pericytes, and then astrocytes to the bottom (i.e. “brain” side) of the membrane, on the opposite side of the membrane from the endothelial cells. Appropriate cell concentrations for pericytes in a BBB model have been established. (Bhatia and Ingber, Nat Biotech, 2014, 32(2):760-72). The thinness of the nanomembranes allows for contact between these cells and the endothelial cells on the top of the membrane. Human cell lines or primary cells are used in each case. This is because many studies done in mice or other lower mammalian models often fail to translate to humans in clinical trials. All cell types mentioned above (including primary human cells) are commercially available. Medium is flowed through both channels at the rates determined in Example 1, so that the shear stress experienced by each cell type contributes to proper development of tight junctions. The barrier is characterized by both TEER and immunohistochemical stains for tight junction proteins, e.g. occludin, claudin-5, and ZO-1. Once the barrier is established within the PIC-BBB, and reaches integrity of similar in vitro models (i.e. >140Ω·cm² for HBECs cocultured with astrocytes), the photonic chip can be utilized for sensing.

To emulate the pathology of AD, AP is introduced into the brain channel. Aβ 1-42 peptide has been shown to preferentially disrupt tight junctions relative to the Aβ 1-40 peptide, and to stimulate production of proinflammatory cytokines by brain endothelial cells. This can be done by injecting AP directly into the bottom channel, but in the interest of modeling AD more closely to what is seen in vivo, in some embodiments, neuronal or astrocytic cell lines that over produce Aβ, are included, as Alzheimer's mutant cell lines are commercially available. Barrier integrity is tracked by quantifying FD40 on the brain side of the barrier (which is being flowed only in the top channel) using anti-FITC antibody-functionalized sensors on the PIC. This allows the time course of barrier disruption can be determined by quantification of protein passage through the barrier with the photonic sensor chip, eliminating the need for electrical measurements. Initial measurements are carried out on the minutes-to-hours scale, then over the course of days and weeks thereafter (primary neurons, immortalized brain endothelial cells, astrocytes, and pericytes, have all been maintained in culture for over three weeks). Although the clinical progression of AD depends on the accumulation of Aβ and tau deposits and consequent neuronal death over the course of years, immune activation on a local scale can be studied on an hours-to-days timescale, allowing this culture system to provide valuable information on AD-related neuroinflammation. Additionally, the early stages of AD, which include these inflammatory processes, are poorly understood, revealing a valuable application for this model system. In addition to measuring Aβ, the PIC is functionalized with antibodies to the cytokines implicated in AD, including IL-1β and IL-6.

In one embodiment of the chip design, it provides 18 rings for sensing (accounting for 2 temperature-control and 4 anti-mIgG control rings) per chip. If the standard deviation for analyte shifts is low enough to indicate the reliability of the shift for each ring (as determined in preliminary experiments), then only one ring is necessary for each analyte. If ring-to-ring variability is high, then two or more rings are used per analyte. This leaves the ability to sense 9 to 18 targets with this embodiment of chip design. IL-1β and IL-6 are tested and quantified according to response curves generated in the lab. Additional targets include TGF-β, TNF-α, and IFNγ, as they are also thought to be involved in AD pathophysiology. The astrocytic protein S100B can also be measured.

The production of cytokines can induce toxic downstream effects on neurons, though the time course of this and its relation to AD pathology is unknown. To examine this question, markers of neuronal health are sensed to test the relationship between cytokine production and neurodegeneration. Specifically, cytochrome c is released from neurons following apoptosis, and can be measured to quantify apoptosis. Aβ aggregation can also be quantified, since Aβ plaques are heavier than monomers, and result in a larger resonance shift.

Several proteins are quantified across a wide time range (minutes to days), identifying their role in AD pathophysiology. Additionally, using different cell types adds complexity to the understanding of how each cell type contributes. This provides new data on how AD progresses, by controlling the factors that make animal models and clinical data difficult to interpret.

The sensitivity of the photonic chip will vary for each protein, based on its affinity for its antibody, and the molecular weight of the protein. For analytes that are difficult to sense, the signal may be amplified by adding a sandwich antibody to the channel containing the sensor. This is not ideal, as one of the advantages of photonic biosensors is their label-free nature, but is used as necessary for certain analytes, particularly lighter peptides such as Aβ.

In these embodiments, therefore, the inventive devices utilize a photonic biosensor chip to simultaneously measure markers of barrier disruption (FD40), cytokine secretion (IL-1β, IL-6, etc.), and neuronal response (cyt c), and determine how these factors interact in real time. This is the first instance of a system combining the advantages of photonic ring resonator biosensors and organ-on-a-chip microfluidic systems, and allows for high throughput testing of neurotherapeutics.

### Example 3

This Example describes the construction and use of an exemplar hToC microfluidic device.

This embodiment of a hToC device combines elements that feature: (1) collagen hydrogel slabs suspended between fluidic compartments and 2) vacuum driven actuators that cyclically stretch the hydrogel in uniaxial fashion. In a current preferred embodiment, the entire device is 40 mm long, 20 mm wide, and ˜3 mm tall, with a collagen gel slab that is 19 mm long, 5 mm wide, and about 500 nm tall. Highly permeable and optically clear silicon nano- and micro-membranes provide fluidic access to the tendon domain while protecting it from destructive flow forces during the introduction or removal of samples.

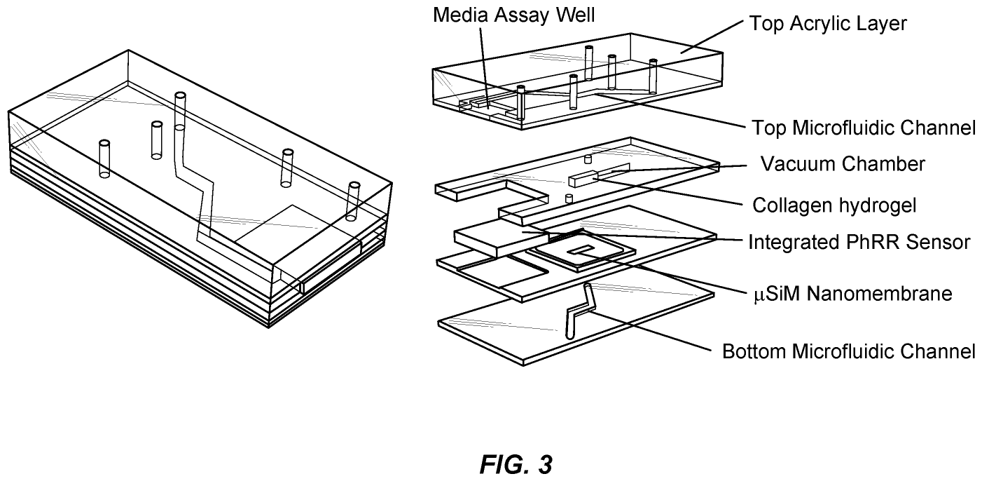

A prototype design is shown in FIG. 3. Referring to FIG. 3, a central channel containing the tendon hydrogel is flanked above and below by fluidic channels containing media, and on a far end by a flexible wall that applies load to the hydrogel by expanding and contracting in response to negative pressure in an adjacent vacuum chamber. A top acrylic (PMMA) housing is used to provide fluidic access to the device. The bottom layer is a glass coverslip, and all other layers are patterned from bioinert pressure sensitive adhesive (“PSA”), with the exception of the membrane spacer layer, which is cut from silicone gaskets. PDMS is preferably avoided for all layers in contact with the culture, as that circumvents artifacts arising from the ability of PDMS to deplete key organic molecules through hydrophobic interactions. Rat-tail type I collagen is pre-mixed with tendon fibroblasts and loaded into the central channel through a loading port to create the tendon hydrogel (dimensions noted above). While suspended collagen gels confined to these dimensions have been shown to support themselves through surface tension, even modest shear from pipetting can cause the gel to flow. Thus, a nanomembrane is disposed beneath the hydrogel to provide support. An endothelial layer is added to the nano- or micromembrane to create the “blood” or “vascular” channel. Other components (such as resident or circulating macrophages, neutrophils, monocytes, leukocytes, mast cells, or combinations of immune cells, with or without other cell types) can be added to this basal compartment to create the “blood” channel side of the hToC.

Computational modeling and experimental testing is used to assure uniaxial strains between 1% and 5% are achieved during cyclic application of loads for over 24 hours. Computational models are done with the MEMS module of COMSOL Multiphysics™, which enables the modeling of interactions between deformable chamber walls and fluids including viscoelastic fluids such as the tendon domain. A geometrically accurate structural model built in SolidWorks™ serves as input into this COMSOL simulation. Experimentally 1 μm fluorescent beads are embedded in collagen matrix prior to gelation to track displacement and strain fields using microscopy. Negative pressure is then applied to the vacuum chamber and monitor deformations using the beads throughout the gel using spinning disk confocal microscopy to build strain maps. Guidance from the mechanical model determines the amplitude of the programmed negative pressure wave.

Tendon fragments typically discarded as surgical waste from hand surgery (primary repair of flexor tendons and tenolysis (surgical release of adhesions)) are obtained from 20 patients. The population of persons for such surgery typically comprises active young individuals (ages 20-40) with a male to female ratio of 5 to 1. Two types of tissues are collected: tissues from acute hand tendon injury repair surgeries (no history of fibrosis; 10 tissues) and tissues with fibrotic adhesions from tenolysis surgeries (fibrosis disease; 10 tissues). The collected tendon tissues are segmented to allocate samples for histology scoring of the pathology, isolation of RNA for next-generation sequencing, and tendon fibroblast isolation using gentle enzymatic digestion and tissue explant outgrowth protocols.

The primary tendon fibroblasts are passaged twice and either cryopreserved for subsequent use in the creation of the tendon hydrogel or transferred to the Upstate Stem Cell cGMP Facility (“USCGF,” Rochester, NY) for the reprogramming and characterization of human induced pluripotent stem cells (“hiPSC”) and the subsequent differentiation into endothelial cells and macrophages. To the best of our knowledge, there exist no reliable protocols to generate tendon fibroblasts from hiPSC. Therefore, these primary cells are used to create the tendon hydrogel. Donor-matched primary cells and hiPSC-derived endothelial cells and macrophages are used in constructing the hToC devices. The hiPSC and their endothelial cells and macrophage-derivatives are reprogrammed using nonintegrating episomal plasmid vectors pCXLE-hOCT4-shP53, pCXLE-hSK and pCXLE-hUL plasmids. Subsequent to hiPSC-reprogramming, multiple clones per donor (at least 3-5) are characterized for expression of pluripotency markers, presence of normal karyotype, and sterility. Given the variable efficiency of different hiPSC-lines to generate specific cell types, the potency of the hiPSC clones is evaluated to select 2-3 clones that consistently differentiate into the desired cell type (endothelial cells and macrophages) for use in experiments. Unused clones are deposited at the WiCell Bank (www.wicell.org/) using their standard operating procedures. Endothelial cells (“ECs”) from hiPSCs are generated in a differentiation protocol where hiPSCs are first differentiated to early mesoderm, followed by hematovascular mesoderm and subsequently EC progenitors. Similar to hiPSC-EC differentiation, hiPSC macrophages (hiPSC-M) are derived using a stepwise protocol that begins with mesoderm induction followed by hematopoietic specification and subsequently myeloid progenitor expansion and macrophage maturation.

Our data in a mouse model demonstrate that, when tendon is injured, the injury site is infiltrated with Csflr+ve macrophages as early as 3 days post injury (“dpi”). Subsequently, activation of α-SMA+ve myofibroblasts is evident even after 28 dpi. In vitro, when tendon fibroblasts are exposed to macrophage-conditioned media, the percentage of myofibroblasts is significantly increased, suggesting that cytokines released by the macrophages activate myofibroblasts. In addition, when tendon fibroblasts are seeded onto axially-constrained hydrogel and treated with TGF-β1, the fibroblasts differentiate into myofibroblasts as evident by the dose-dependent increase in α-SMA gene (Acta) expression and hydrogel lateral contraction. Furthermore, the relative expression of ECM genes is significantly increased with increased doses of TGF-β1, without appreciable changes in MMP gene expression, indicating the suitability of the collagen hydrogel to model fibrotic scar in tendon injury.

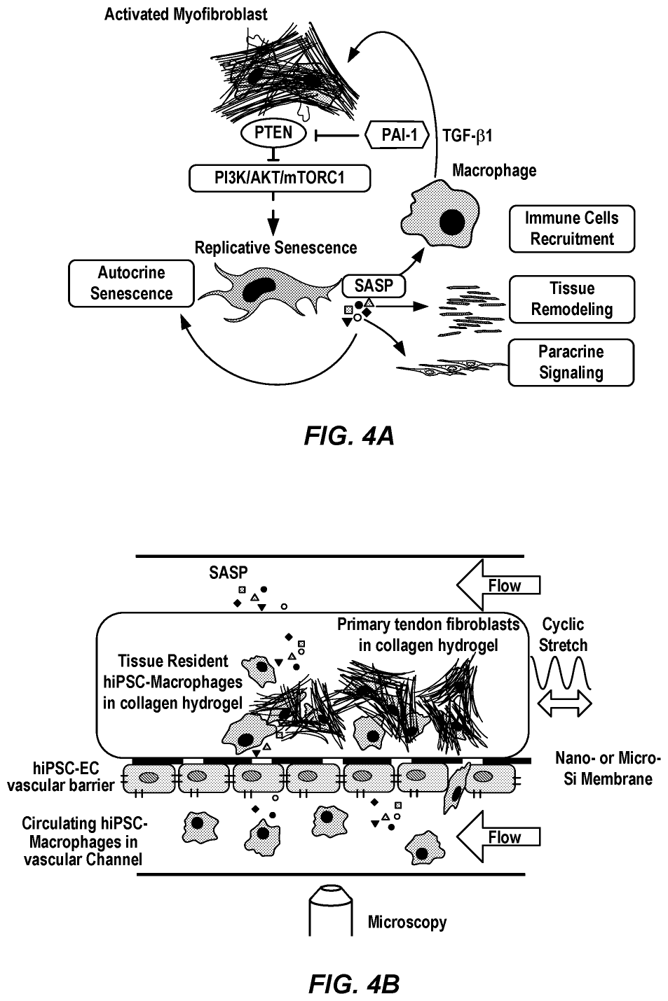

FIG. 4A shows a proposed pathobiologic model and druggable targets in chronic inflammation and tendon fibrosis following tendon injury. FIG. 4B shows a schematic representation of the experimental setup on the hToC to investigate the role of tissue-resident and circulating macrophages in activating the differentiation of myofibroblasts and the SASP-induced senescence by mTORC1 signaling.

To simulate the microphysiological environment of an injured tendon, collagen hydrogel is cast in its specialized chamber in the hToC on the top side of a nano- (60 nm) or micro- (8 μm) porous Si ultrathin membrane (SiM) to model paracrine signaling only and macrophage extravasation from circulation, respectively. The collagen is seeded with primary tendon fibroblasts and donor-matching hiPSC-derived macrophages (hiPSC-M). The cell seeding density is 50,000 cells/ml, based on experimentally measured values in injured tendons. The collagen hydrogel is cyclically stretched to 1% at 1 Hz. The bottom side of the porous SiM will be seeded to confluence with hiPSC-derived endothelial cells (hiPSC-EC) to create a vascular endothelial barrier. Vybrant™ Dil-labeled circulating hiPSC-M (Vybrant™ Dil, Thermo Fisher Scientific, Waltham, MA) are flowed through the bottom microfluidic channel to image transmigration events. The hiPSC-M are naïve (Mφ), classically activated (M1), or alternatively activated (M2). Perturbation of mTORC1 and mTORC2 signaling is accomplished by introducing experimental selective inhibitors of AKT (MK-2206), PI3K (PF-04691502), and mTOR (AZD8055, CZ415, Torin1) (see, Woodcock et al., Nat Commun., 2019;10(1):6. Epub 2019/01/04). Proper controls are used, including macrophage-free setup with or without TGF-β1 treatment (10 ng/ml) and mechanical loading. Readouts: Live fluorescence microscopy is used to image the transmigration of labeled circulating hiPSCM. Endpoint measurements include the proliferation and differentiation of myofibroblasts using Ki67 and α-SMA IHC staining, the induction of fibroblast senescence using X-gal and γH2AX immunostaining, the activation of mTOR using immunostaining, and the quantification of secretion of SASP (e.g. CXCL10, CCL2, CCL3, TNF-α, IL-1B, IL-6, IL-10, IL-17) using multiplex ELISA. mTORC1 signaling is assessed by lysing the hydrogel and performing Western blot analysis to probe for total and phosphorylated AKT, S6, mTOR4EBP1, 4E-BP1, and NDRG1 with proper loading control (β-actin) (see, Woodcock et al., supra.). In addition to the ELISA SASP determination, media is retained and frozen to enable later analysis by mass spectrometry if further identification of proteomic biomarkers is deemed desirable.

Quantifiable outcomes (SASP concentration) are compared using ANOVA with Bonferroni-corrected multiple comparisons. SASP changes are correlated with the decrease in myofibroblast and senescent cell numbers to determine the most sensitive SASP to mTOR inhibitors. The sample sizes are set arbitrarily to use hToC devices constructed with cells from 5 unique donors, and experiments are done in triplicate for each donor.

Eight-plex arrays are prepared to demonstrate the compatibility of PhRR and 2DPhC for multiplex arrays by confirming their ability to detect target proteins doped into cell culture media at appropriate concentrations. FIG. 5 shows the layout of an exemplar PhRR chip suitable for use for the demonstration. Each bus waveguide addresses pairs of ring resonators; in each case, one ring is functionalized with a control antibody (anti-fluorescein) to correct for nonspecific binding, while the other is functionalized with an antibody for one of the 8 SASP targets. One paired set of PhRRs in each bank includes a single ring under oxide (not exposed to the environment) as a thermal control, and another which is also derivatized with anti-fluorescein. 2DPhC arrays are structured in the same way; for these arrays, a data analysis method (Baker and Miller, Opt Express. 2015(23):7101-10) is used to compare defect- and band-edge resonance shifts to provide enhanced discrimination of specific vs. nonspecific binding. In both cases, antibodies are immobilized on the surface using surface chemistry and piezoelectric spotting methods. (Yadav et al., Mat Sci Eng C., 2014, (35):283-90; Zhang et al., Anal Chem., 2018; 90(15):9583-90. Epub 2018/07/10.)

In principle, PhRR and 2DPhC arrays do not require “leave one out” cross-validation testing for cross-reactivity as is commonly done for Luminex and other labeled assays do since each sensor element operates essentially independently, i.e. there is no sandwich antibody to cross react. In our experience, however, specificity of antibodies used for label-free assays is not always absolute, and thus confirmation of specificity is still required. If a particular antibody shows crossreactivity, it is replaced with one of the many other commercially available antibodies for the target molecules. Data generated thus far shows the ability to routinely detect representative cytokines at 100 pg/mL under microfluidic flow. This level is sufficient for the hToC since cytokine concentration near the tissue model are substantially higher than is observed in serum.

### Example 4

This Example describes integrating a PIC sensor chip into a human Tendon-on-Chip device.

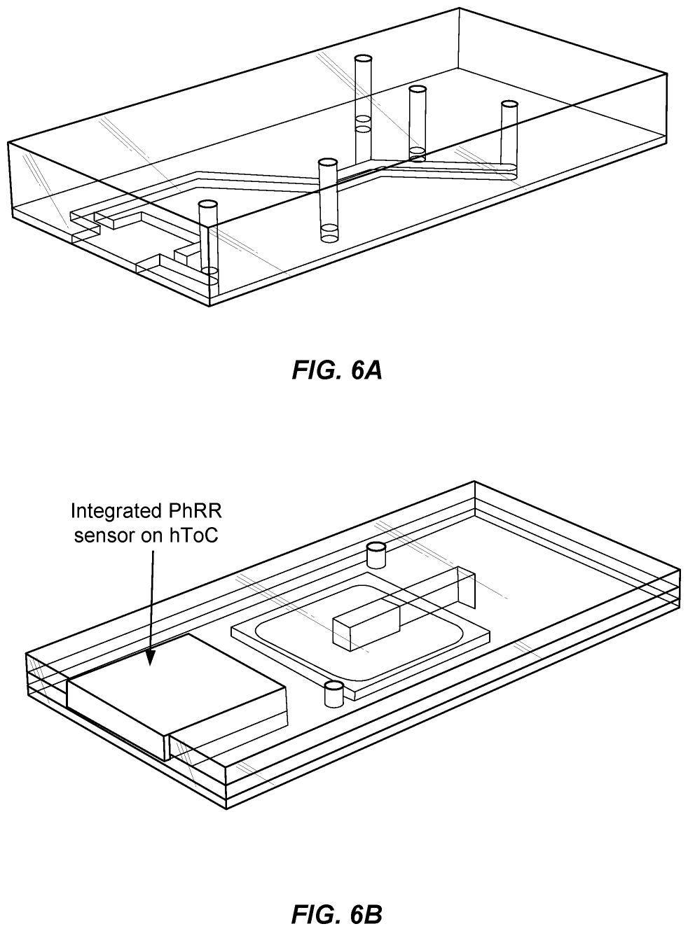

Building on the hToC platform described in the previous Example, PhRR and 2DPhC sensor chips are integrated with the hToC platform as shown schematically for PhRR in FIG. 6, which shows a schematic of the hToC device. The multilayer assembly has been modified in the schematic to accept a photonic chip at one end in the same layer as the nanomembrane support chip. In this embodiment, the placement of the photonic chip at the edge of the device enables facile coupling to an optical fiber array.

After confirmation that PhRR sensors and 2DPhC sensors function as observed in their initial validation experiments while integrated in the hToC in the absence of cells, sensor performance is tested with the full tissue model in place. Sensor performance is assessed at baseline levels of SASP targets and following appropriate stimulation to induce elevated SASP levels. Each design is benchmarked against ELISA assays. The analytical performance of the PhRR and 2DPhC sensor arrays is determined. While each chip is expected to be useful for its intended purpose, additional factors are relevant for commercial sales, including performance over time (i.e. susceptibility to fouling), chip-to-chip reproducibility, and sensor regeneration performance.

For some cytokines or other analytes, the analytical performance metrics (particularly lower limits of quantitation) may be insufficient. For such analytes, the PhRR or 2DPhC sensor chip protocol is modified to include externally added sandwich (reporter) antibodies at specific time points. The reporter antibody substantially improves sensitivity for analytes present in only small quantities, since PhRRs and 2DPhCs are essentially mass sensors. Alternatively, if cases in which the release of a particular cytokine is too abundant to quantify with PhRRs or 2DPhCs sensors, a calibrated amount of the primary antibody for that cytokine can be flowed through the microfluidic channel to convert the assay to a competitive, less-sensitive format. If alignment of the fiber arrays used for optical in/out (“I/O”) proves to be challenging for production when sensors are integrated into the hToC system, sensor chips with optical fibers permanently attached (fiber pigtailing) can be used.

### Example 5

This Example describes the utility of using PIC sensor chips in a human Tendon-on-Chip device for pre-clinical screening.

The inventive hToC devices are expected to be useful in pre-clinical studies to demonstrate the efficacy and safety of candidate therapies clinically investigated for lung fibrosis but not currently used for tendon fibrosis. The devices are expected to provide a pre-clinical proof of concept and de-risk future clinical trials, thereby notably reducing the cost and uncertainty of moving forward with repurposing these therapeutic agents.

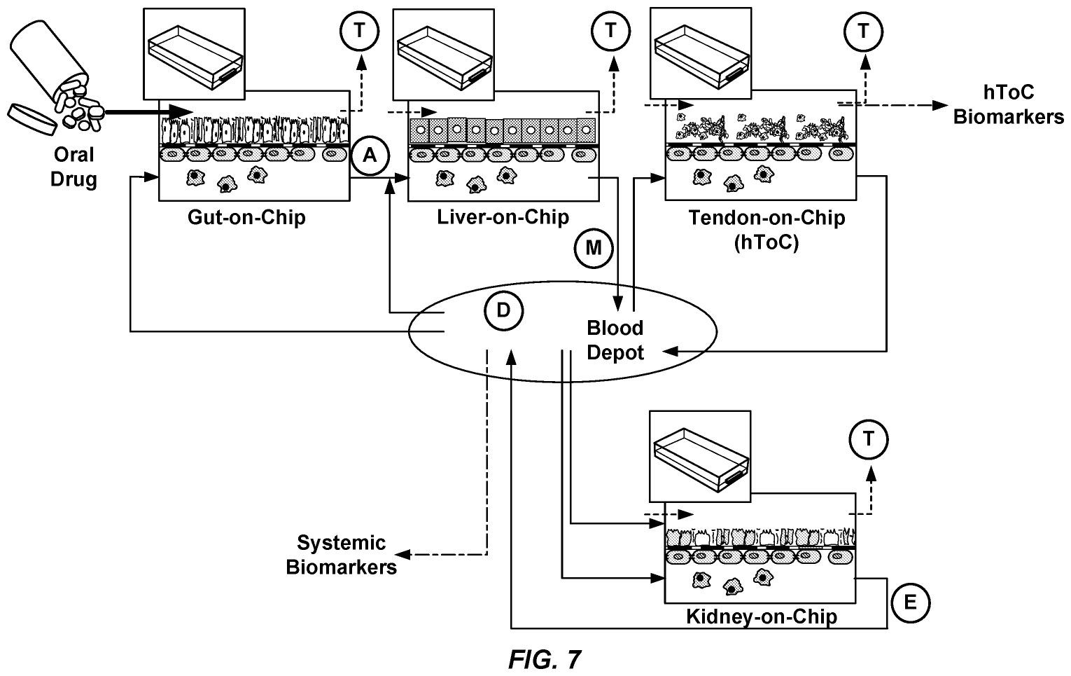

The patient-centric hToC model discussed above will be used to evaluate the effectiveness of the FDA-approved mTOR inhibitors sirolimus and everolimus, which are being investigated in various fibrosis pathologies, and compare them to non-disease modifying steroids, such as prednisone. The hToC model can also be linked system with other organ hMPS to perform proof-of-concept ADMET (an acronym for “Absorption, Distribution, Metabolism, Excretion, and Toxicity”) studies, as shown schematically in FIG. 7. Furthermore, the hToC will also be used for drug screening of a library of mTOR inhibitors and synolytics.

The hToC system can also be used to determine the doses that produce the desired pharmacologic effect of mitigating fibrosis with reasonable safety outcomes based on the viability of the cells in the hMPS. To do this, a “Virtual Clinical Trial-on-a-Chip” is used for dose-escalation studies to determine the minimum required dose for efficacy and maximum dose for safety based on outcomes quantifying fibrosis and toxicity in the system, as detailed below. These doses determine the target plasma concentrations for the indications tested and will provide useful in formation in designing dosing recommendations of drug candidates for tendon pathologies. The clinical doses of these drugs and ED₁₀ or ED₅₀ values estimated in rodents in the literature is expected to be supraphysiologic for the hToC system. Accordingly, the dosing schedule informed by measured plasma drug concentration of these drugs or target whole blood trough concentrations (provided by pharmaceutical makers as general guidelines) shown in Table 1 will be adjusted as needed.

TABLE 1

| | | Clinical | | |
|---|---|---|---|---|
| | | dose mg/day | Target whole | |
| | | (based on | blood trough | Dose |
| | | oral tablet | concentrations | escalation |
| | ED50 | concen- | (plasma | schedule, |
| Drug | mg/kg | trations) | concentrations) | ng/ml |
| Sirolimus | 0.28-1.6 | 0.5-2 | 16-24 ng/mL | 0 (control), |
| | | | | 0.1, 1, 10, 100 |
| Everolimus | ~0.1-2.38 | 2.5-10 | 5-15 ng/mL | 0 (control), |
| | | | | 0.1, 1, 10, 100 |
| Prednisone | | 1-50 | 2-12 ng/mL | 0 (control), |
| | | | | 0.1, 1, 10, 100 |

### Example 6

This Example describes the integration of PIC sensor chips in human Tendon-on-Chip devices with devices modeling organ systems to provide pre-clinical information on absorption, distribution, and kinetics.

The hToC platform can be linked with commercially available OOC chips of intestines, liver, and kidney, organs that could affect a drug's Absorption, Distribution and bio availability, Metabolism, and Excretion (ADME) kinetics, in a microfluidic circuit, or can be used with organ chips described in the present disclosure. The candidate drugs for tendon fibrosis (sirolimus, everolimus, and prednisone) discussed in the preceding Example are administered to the intestine OOC intestinal lumen channel to simulate orally administered drugs. FIG. 7 is an illustration showing the use of an exemplar organ chip, a hToC chip, in such a system. The letters in circles are from the acronym ADMET (which, as mentioned above, is an acronym for “Absorption, Distribution, Metabolism, Excretion, and Toxicity”), and indicate what aspect of drug pharmacodynamics is being evaluated at each step. Macrophage-laden media flow from a central “blood” depot is circulated into the vascular channels of the hMPS devices according to the flow circuit schematically shown in the Figure. The system parameters, including drug dose, flow rate, and cell numbers (densities) are allometrically scaled to achieve a blood depot concentration in the range of Target Whole Blood Trough Concentrations for these drugs as a starting point. The integrated system allows simulating oral drug delivery and systemic ADME, while the hToC chip senses the SASP biomarkers and determines the efficacy of treatments. Sampling the media allows measuring drug concentrations to determine the ADME parameters in the integrated system. Off-target toxicity can be determined by sampling the media from each hMPS to measure fortilin and caspase 3 as a marker of cell death and apoptosis.

### Example 7

This Example describes the integration of PIC sensor chips in a human Tendon-on-Chip for use in drug screening.

Use of integrated multiplex photonic sensing in the hToC provide real-time information useful for enabling rapid drug screening. To that end, we will screen the DiscoveryProbe™ PI3K/Akt/mTOR Compound Library (APExBIO) to identify hits effective in inhibiting mTOR based on measured SASP in the hToC Minimum effective doses of identified hits will be further determined by microscopy assessment of the differentiation of myofibroblasts using α-SMA IHC staining and fibroblast senescence using X-gal and γH2AX IHC staining. Further, Western blot analysis to probe for total and phosphorylated AKT, mTOR, S6, 4E-BP1, and NDRG1 will demonstrate the direct effects on mTOR activation. The toxicity of the identified hits will be assessed to determine the maximum safe doses by probing for cell viability using the CellTox™ Green Cytotoxicity Assay and by measuring levels of fortilin and caspase 3 as markers of cell death and apoptosis in circulating media.

### Example 8

This Example describes the use of an exemplar device of the invention with an “on-board” photonic ring sensor chip to monitor in real time the secretion of cytokines from cells of a human cell line in response to a chemical of interest.

Cytokine secretion in response to pathogenic stimuli is an important immune function. Understanding these processes is important for developing treatment for various infectious diseases. Lipopolysaccharide (“LPS”) is an important marker for gram-negative bacterial pathogens which is encased in the cell wall of these infectious agents. Many mammalian cell types are known to respond to exposure to LPS by secreting cytokines. Human bronchial epithelial cells of the 16HBE cell line have previously been shown by others to secrete the cytokines IL-1β and IL-6 in response to contact with LPS (Shirasaki et al., Sci Rep., 2014; 4(1):1-8. doi:10.1038/srep04736). This Example was performed to conduct a similar experiment, using an exemplar microfluidic chip device bearing photonic ring sensors, to see how the results using an “on-board” chip compared with the more laboriously-obtained results reported by Shirasaki et al.

16HBE cells were seeded on the membrane of the exemplar device and allowed to adhere to the membrane for 3 hours. The device was then connected to a peristaltic pump, which contacted the cells with Dulbecco's Modified Eagles' Medium (DMEM) at a low flow rate (˜30 μL/min) overnight. The next day, the device was set on a temperature-controlled stage (37° C.) and DMEM doped with LPS (at 1 μg/mL) was flowed over the cells (through the top channel) at 30 μL/min for 3 hours. Spectra were measured continuously to observe any shifts in resonance of the photonic ring resonators over time.

The results are shown in FIGS. 1F and 1G. FIG. 1F is a graph with a Y axis showing the wavelength of light resonant in the test and the control ring sensors, with longer wavelengths towards the top, and an X axis showing time in seconds following exposure of the cells to LPS. The top two lines show the raw peaks of wavelength corresponding to the test photonic ring sensors, which are functionalized, in the graph shown, with an anti-IL-6 (a-IL-6) antibody (top line), and control photonic rings to which bovine serum albumin had been covalently bound (second line from top). As the control rings have not been functionalized with an antigen-specific antibody, they are not expected to specifically bind any compounds in the media. Binding of the cytokine to an antibody attached to a test ring sensor bearing it causes a shift to the red (towards the top of the Y axis) in the wavelength in the ring sensor. The two dark lines at the bottom of the graph show peaks that higher-order (i.e., lower wavelength) resonant wavelengths of the rings, which result in identical shifts. (As persons of skill will appreciate, such resonant wavelengths are a regular aspect of using photonic ring sensors and are not considered as part of the experimental results.) The references to the lines in the rest of this discussion therefore refers to the top two lines shown on the graph.

As the media flowing from the cells passes over the ring sensors, there is initially some non-specific binding to both the test and the control rings, as can be seen by the slight upward shift of both the lines on the far left of the graph. FIG. 1F is divided vertically by a dark line, representing the time point at which, based on Shirasaki et al., it was expected that cells contacted with LPS would begin secreting IL-6. As the lines for the test rings and the control rings proceed to the right after that point, the line for the test rings can be seen to shift upward as IL-6 is secreted from cells and binds to the functionalized rings.

FIG. 1G presents graphs showing the results of subtracting the shifts in the control rings (indicating non-specific binding) from that of the control rings for two cytokines, IL-6 (left graph) and IL-1B (right-hand graph). Each graph shows the results from four control-test ring pairs over the course of ˜3 hours. For the reader's attention, it is noted that the scale of the two graphs is different, with much smaller quantities of cytokine being detected in the right-hand graph. It is believed that, because the sensor rings are so close physically to the cells, the rings are able to detect small changes in concentration of the analyte they have been designed to detect (for example, by being functionalized with an antibody or other molecule that specifically binds the analyte which the practitioner wishes to detect.)

The initial flat region in FIG. 1G shows no response, as the cells require some time to alter their protein expression in response to LPS. The increate beginning at about 70 minutes and then continuing to the end of the time shown is due to the detection by the test rings of cytokines secreted from the 16HBE cells in response to continuous stimulation with LPS. The results are in excellent agreement with the results reported by Shirasaki et al., supra, and show the ability to use on-board photonic ring sensor chips to detect in real time changes in analytes secreted by cells in response to changes in experimental conditions.

### Example 9

This Example describes the detection of the secretion of a number of cytokines from cells of a human cell line under a series of experimental conditions.

In all of the studies reported in this Example, tendon cells known as “tenocytes” were cultured in what a channel present in an acrylic structure shown as the bottom channel in the exploded view of FIG. 8D, sealed at the base with a sheet of transparent cyclic olefin copolymer (“COP”), as depicted in the exploded view of that Figure and labeled as “COP Imaging Layer.” (For convenience, the combination of the acrylic piece with a cutout section defining the bottom channel, and sealed at the bottom with COP bottom, will be referred to in the rest of this Example as the “bottom-sealed bottom channel,” and references to the “bottom channel” will refer to the acrylic piece whose sides define the channel itself.) In some of the studies, tenocytes were incubated in a collagen matrix hydrogel in the bottom-sealed bottom channel, without use of a top modular unit, and with or without a second cell type, M0 monocytes, present in the hydrogel. The acrylic piece defining the bottom channel in this embodiment, which was designed for use with tenocytes in a collagen hydrogel, includes two crosspieces spanning the width of the channel. Tenocytes, which are derived from tendon, contract, and the crosspieces, called “hydrogel anchors” in FIG. 8D, provide some additional support for the hydrogel when it is pulled on by the tenocytes. In some of the studies, the tenocytes were cultured in the hydrogel with M0 monocytes for a period of days, a top module was then added with a further cell type, the combined modular device was co-cultured for 72 hours, and the supernatant examined for the presence of the cytokines of interest.

In a first set of studies, tenocytes (“TC”) were cultured in collagen hydrogel in the bottom-sealed bottom channel for seven days in “X-VIVO 10” media (Lonza Corp., Basel, Switzerland), either without TGF-β1 (“TC−TGF-β1”) or with TGF-β1 added at 10 ng/mL (“TC+TGF-β1”). Supernatant samples were taken and the levels of eight cytokines, MCP-1, IL-6, CCL3, IL-10, CXCL10, IL-1β, TNF-α, and IL-17, were measured using a magnetic bead-based multiplex assay (Human Luminex Discovery Assay, catalog no. LXSAHM-08, Luminex Corp., Austin, TX) per the manufacturer's instructions. The results are shown in FIG. 9D, described in the legend as the results labeled “Mono-culture.”

In a second set of studies whose results are reported in FIG. 9D, reported under the legend “Co-Culture,” tenocytes (“TC”) were co-cultured with M0 monocytes (“M”) in a collagen hydrogel in a bottom-sealed bottom channel for seven days in X-VIVO 10 media. Supernatant samples were taken at three intervals after seeding of the collagen hydrogel with the TC and M: 24 hours after seeding (shown in the legend as “D1”), on day 4 (“D4”), and at 7 days (“D7”). Cytokine levels were measured for each supernatant sample as described in the previous paragraph.

In a third study whose results are reported in FIG. 9D, reported under the legend “Tri-Culture,” tenocytes (“TC”) were co-cultured with M0 monocytes (“M”) in collagen hydrogel in a bottom-sealed bottom channel for seven days in X-VIVO 10 media supplemented with 10% fetal bovine serum (“10% FBS;” in later studies, the FBS was omitted). On the eighth day, endothelial cells (“EC,” cell line HUVEC) were added to a top module with a “dual-scale” membrane (in this case, one having both micro- and nano-sized pores) disposed at the bottom of top module and allowed to form a monolayer in the same media as described above. Referring to FIG. 8B, the bottom-sealed bottom module was manufactured with an adhesive coating the top of the gray area defining the bottom channel, which adhesive was covered with a protective film until use. After the monolayer formed on the dual-scale membrane in the top module, the protective film covering the top surface of the bottom channel was removed to expose the adhesive and the top module was pressed gently onto the bottom-sealed bottom module, with the adhesive both adhering the top module to the bottom-sealed bottom channel and creating a water-proof seal around the sides of the area at which the top module met the bottom-sealed bottom channel (for clarity, it is noted that the top module was fluidly connected with the bottom-sealed bottom channel through the dual-scale membrane). Returning to the study reported in FIG. 9D, supernatant samples were taken 72 hours after the top module was joined to the bottom-sealed bottom channel and cytokine levels measured by the Luminex magnetic bead assay as described earlier in this Example. As noted, the results from the study are set forth in FIG. 9D, represented by the symbol labeled in the legend of FIG. 9D as “Tri-Culture M/TC/EC 10% FBS.”

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A microfluidic device for providing real-time information on analytes, said device comprising:
(a) a first microchannel fluidly connected to a port on an exterior of said device, and having a length, a first end, and a second end,
(b) an ultrathin membrane having nanopores, mesopores, micropores, or a combination of two or more of these, said ultrathin membrane having a first side and a second side, wherein said first side of said membrane is fluidly connected through said first microchannel to said port on said exterior of said device,
(c) a second microfluidic channel, which second microfluidic channel faces said ultrathin membrane and is fluidly connected to receive any fluid coming through nanopores, mesopores, micropores, or combinations thereof of said ultrathin membrane, and,
(d) a first photonic integrated circuit sensor (“PIC sensor”) disposed in said first microchannel or in said second microchannel, which first PIC sensor is functionalized to detect the presence of a first analyte of interest in fluid in said first microchannel or said second microchannel, respectively.

2. The microfluidic device of claim 1, wherein said ultrathin membrane is a nanoporous membrane, a mesoporous, a microporous membrane, has a combination of any two pore sizes selected from nanopores, mesopores, and micropores, or has nanopores, mesopores, and micropores.

3-5. (canceled)

6. The microfluidic device of claim 1, wherein said ultrathin membrane is of silicon nitride.

7-8. (canceled)

9. The microfluidic device of claim 1, further comprising a second PIC sensor, which second PIC sensor is disposed in said first microchannel or in said second microchannel, and is functionalized to detect the presence of a second analyte of interest in fluid in said first microchannel or said second microchannel, respectively.

10. (canceled)

11. The microfluidic device of claim 1, further comprising an outlet in said second microchannel to allow fluids in said second microchannel to exit the device.

12-17. (canceled)

18. The microfluidic device of claim 1, configured to allow said first PIC sensor to be exchanged by sliding said first PIC sensor out and sliding a fresh PIC sensor in.

19. (canceled)

20. The microfluidic device of claim 1, wherein cells of a first cell type are disposed on said first side of said ultrathin membrane.

21-23. (canceled)

24. The microfluidic device of claim 20, wherein said cells of a first cell type disposed on said first side of said ultrathin membrane are tendon fibroblasts, and said device is configured to provide uniaxial stress to said tendon fibroblasts.

25-27. (canceled)

28. A method of detecting if a first analyte of interest has been released from cells of interest or through an interaction between two or more types of cells of interest, said method comprising:
(a) obtaining a microfluidic device comprising
(i) a first microchannel fluidly connected to an exterior of said device, and having a length, a first end, and a second end,
(ii) an ultrathin membrane having nanopores, mesopores, micropores, or a combination of two or more of these, said ultrathin membrane having a first side and a second side, wherein said first side of said ultrathin membrane is fluidly connected to said first microfluidic channel,
(iii) a second microfluidic channel, which second microfluidic channel is fluidly connected to said second side of said ultrathin membrane, and,
(iv) a first photonic integrated circuit sensor (“PIC sensor”) fluidly connected to fluid in said first microchannel or said second microchannel, wherein said first PIC sensor is functionalized to change a detectable property of said first PIC sensor if a selected first analyte is present in fluid with which said first PCT sensor is in contact, thereby signaling said first analyte is present in said fluid,
(b) disposing cells of a first cell type of interest on said first side of said ultrathin membrane,
(c) allowing fluid in contact with said cells of said first cell type of interest on said first side of said ultrathin membrane to contact said first PIC sensor, and
(d) detecting any signal from said first PIC sensor indicating the presence of said first analyte of interest in said fluid,
thereby detecting whether said first analyte of interest has been released from cells of interest or through an interaction between two or more types of cells of interest.

29. The method of claim 28, wherein said ultrathin membrane is a nanoporous membrane, a mesoporous membrane, a microporous membrane, has a combination of any two pore sizes selected from nanopores, mesopores, and micropores, or has nanopores, mesopores, and micropores.

30-32. (canceled)

33. The method of claim 28, wherein said ultrathin membrane is of silicon nitride.

34. (canceled)

35. The method of claim 28, wherein said first PIC sensor is disposed on a layer in said device holding said ultrathin membrane.

36-43. (canceled)

44. The method of claim 28, wherein said functionalization of said PIC sensor is by covalently attaching to said first PIC sensor an antibody that specifically binds said first analyte of interest.

45. (canceled)

46. The method of claim 28, wherein said cells of a first cell type disposed on said first side of said ultrathin membrane are epithelial cells or brain endothelial cells.

47-49. (canceled)

50. The method of claim 28, wherein said cells of a first cell type disposed on said first side of said ultrathin membrane are tenocytes.

51. (canceled)

52. The method of claim 50, wherein said device is configured to provide uniaxial stress to said tendon fibroblasts.

53. (canceled)

54. A modular microfluidic device, said modular microfluidic device comprising:
(a) a first module having a length, a width, a top, and a bottom, said first module comprising
(i) a well or a first microchannel, said well or first microchannel fluidly connected to an exterior of said device, and,
(ii) an ultrathin membrane having nanopores, mesopores, micropores, or a combination of two or more of these, said ultrathin membrane having a first side and a second side, wherein said first side of said membrane is fluidly connected to said bottom of said well or of said first microchannel, and
(b) a second module, having a length, a width, a top, and a bottom, wherein said top of said second module has a length and a width configured to mate with said bottom of said first module, said second module comprising a second well or second microfluidic channel fluidly connected to said top of said second module, and positioned to fluidly connect to said ultrathin membrane of said first module when said first module is placed on top of said second module.

55. The modular microfluidic device of claim 54, wherein said bottom of said first module has an exterior surface and said top of said second module has an exterior surface, wherein said exterior surface of said bottom of said first module and said exterior surface of said top of said second module are configured to contact each other when said first module is placed on top of said second module.

56. The modular microfluidic device of claim 55, wherein said exterior surface of said top of said second module bears an adhesive.

57-58. (canceled)

59. The modular microfluidic device of claim 54, wherein said well or said microchannel in said second module has at least one crossbar spanning a dimension of said well or said microchannel.

60. The modular microfluidic device of claim 54, wherein said bottom of said second module is covered with a transparent material allowing viewing into said well or said microchannel of said second module.

61. (canceled)

62. The modular microfluidic device of claim 54, wherein said ultrathin membrane is a nanoporous membrane, a mesoporous membrane, a microporous membrane, or has a combination of nanopore and mesopores, of nanopores and micropores, of mesopores and micropores, or of nanopores, mesopores, and micropores.

63-65. (canceled)

66. The modular microfluidic device of claim 54, wherein said ultrathin membrane is of silicon nitride.

67. The modular microfluidic device of claim 54, further comprising an outlet in said second module allowing fluids in said device to exit.

68. The modular microfluidic device of claim 54, having a first photonic integrated circuit sensor (“PIC sensor”) functionalized to detect presence of a first analyte of interest, which first PIC sensor is fluidly connected to said well, to said microchannel of said first module or to said well or said microchannel of said second module, or to both said well or said microchannel of said first module and to said well or said microchannel of said second module.

69-70. (canceled)

71. The modular microfluidic device of claim 68, wherein said functionalization of said first PIC sensor is by covalently attaching to said PIC sensor an antibody that specifically binds said first analyte of interest.

72. (canceled)

73. The modular microfluidic device of claim 69, further comprising a second PIC sensor functionalized to detect a second analyte of interest, which second PIC sensor is fluidly connected to said well or said microchannel of said first module, to said well or said microchannel of said second module, or to both said well or said microchannel of said first module, and to said well or said microchannel of said second module.

74. (canceled)
